(19) United States
(12) Patent Application Publication
(10) Pub. No.: US 2016/0168335 A1
(43) Pub. Date: Jun. 16, 2016

(54) ALKOXYLATED HUMUS MATERIAL COMPOSITIONS AND METHODS OF MAKING SAME

(71) Applicant: HALLIBURTON ENERGY SERVICES, INC., Houston, TX (US)

(72) Inventors: Kenneth W. POBER, Houston, TX (US); Cato R. MCDANIEL, The Woodlands, TX (US)

(21) Appl. No.: 14/902,665

(22) PCT Filed: Jul. 31, 2013
(86) PCT No.: PCT/US2013/052951

§ 371 Date: Jan. 4, 2016

(51) Int. Cl. C08H 7/00; C07D 265/38; C07B 41/04; C07C 63/40; C07C 51/367; C08G 65/26; C07C 43/02; C07C 41/02; C07G 1/00

(52) U.S. Cl. CPC C08H 6/00; C07C 41/02; C07D 265/38; C07B 41/04; C07G 1/00; C07C 51/367; C08G 65/2696; C07C 43/02; C07C 63/40

(57) ABSTRACT

A method of alkoxylating a humus material comprising heating a reaction mixture comprising a humus material, a C3+ cyclic ether, a catalyst and an inert reaction solvent, and recovering a C3+ alkoxylated humus material from the reaction mixture. A method of alkoxylating a humus material comprising heating a reaction mixture comprising a humus material, a C3+ cyclic ether, a catalyst and an inert reaction solvent to a temperature of from about 130° C. to about 170° C., wherein the humus material comprises leonardite, the C3+ cyclic ether comprises propylene oxide, and the inert reaction solvent comprises xylene, and recovering a C3+ alkoxylated humus material from the reaction mixture. A C3+ alkoxylated humus material.

### BACKGROUND

This disclosure relates to methods of producing chemically modified humus materials. More specifically, it relates to methods of producing alkoxylated humus materials.

Humus materials are readily available and abundant across the planet. The use of a specific humus material in an application will depend on the physical and chemical properties of the humus material. Generally, the physical and chemical properties of the humus materials can be modulated by chemical modification of the humus materials, such as for example alkoxylation of humus materials. Thus, there is an ongoing need to develop and improve methods for producing chemically modified humus materials, e.g., alkoxylated humus materials.

### SUMMARY

Disclosed herein is a method of alkoxylating a humus material comprising heating a reaction mixture comprising a humus material, a C3+ cyclic ether, a catalyst and an inert reaction solvent, and recovering a C3+ alkoxylated humus material from the reaction mixture.

Also disclosed herein is a method of alkoxylating a humus material comprising heating a reaction mixture comprising a humus material, a C3+ cyclic ether, a catalyst and an inert reaction solvent to a temperature of from about 130° C. to about 170° C., wherein the humus material comprises leonardite, the C3+ cyclic ether comprises propylene oxide, and the inert reaction solvent comprises xylene, and recovering a C3+ alkoxylated humus material from the reaction mixture.

Further disclosed herein is a C3+ alkoxylated humus material.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques below, including the exemplary designs and implementations illustrated and described herein, but may be modified within the scope of the appended claims along with their full scope of equivalents.

Disclosed herein are C3+ alkoxylated humus materials (CAHMs) and methods of making same. In an embodiment, the CAHMs may be obtained by heating a reaction mixture comprising a humus material, a C3+ cyclic ether, a catalyst and an inert reaction solvent. In an embodiment, the reaction mixture may be heated in a substantially oxygen-free atmosphere to yield the CAHMs. In an embodiment, CAHMs of the type described herein may be advantageously used as additives in fluids or compositions suitable for wellbore servicing operations.

In an embodiment, the reaction mixture comprises a humus material. In an embodiment, the humus material references a brown or black material derived from decomposition of plant and/or animal substances. Generally, humus represents the organic portion of soil that will not undergo any further decomposition or degradation, and which comprises complex molecules resembling or incorporating at least a portion of a humic acid-like structure. In an embodiment, the humus material may be comprised of a naturally-occurring material. Alternatively, the humus material comprises a synthetic material, such as for example a material derived from the chemical modification of a naturally-occurring material. Alternatively, the humus material comprises a mixture of a naturally-occurring and synthetic material.

In an embodiment, the humus material comprises brown coal, lignite, subbituminous coal, leonardite, humic acid, a compound characterized by Structure I, fulvic acid, humin, peat, lignin, and the like, or combinations thereof.

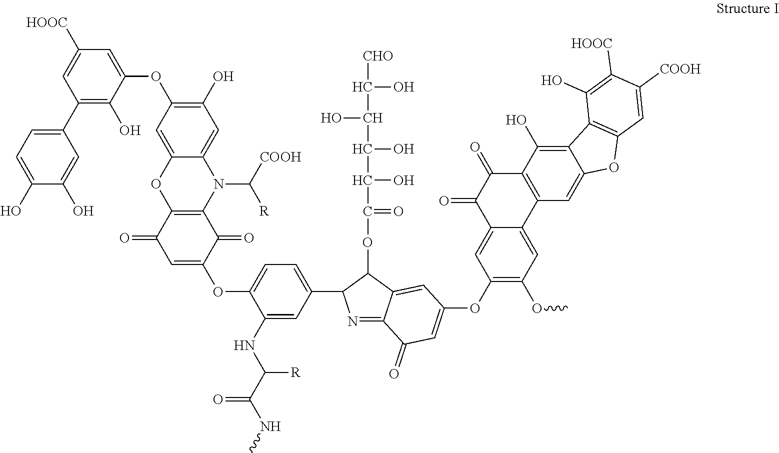

The wavy lines in Structure I represent the remainder of the molecule (e.g., a humic acid molecule).

In an embodiment, the humus material comprises brown coal. Brown coal generally comprises a broad and variable group of low rank coals characterized by their brownish coloration and high moisture content (e.g., greater than about 50 wt. % water, by weight of the brown coal). Brown coals typically include lignite and some subbituminous coals. The coal ranks as referred to herein are according to the U.S. Coal Resource and Classification System.

In an embodiment, the humus material comprises lignite. Lignite is generally a soft yellow to dark brown or rarely black coal with a high inherent moisture content, sometimes as high as about 70 wt. % water, but usually comprises a water content of from about 20 wt. % to about 60 wt. %, by weight of the lignite. Lignite is considered the lowest rank of coal, formed from peat at shallow depths, with characteristics that put it somewhere between subbituminous coal and peat.

In an embodiment, the humus material comprises subbituminous coal. Subbituminous coal, also referred to as black lignite, is generally a dark brown to black coal, intermediate in rank between lignite and bituminous coal. Subbituminous coal is characterized by greater compaction than lignite as well as greater brightness and luster. Subbituminous coal contains less water than lignite, e.g., typically from about 10 wt. % to about 25 wt. % water, by weight of the subbituminous coal.

In an embodiment, the humus material comprises leonardite. Leonardite is a soft waxy, black or brown, shiny, vitreous mineraloid that is associated with near-surface mining. Leonardite is an oxidation product of lignite and is a rich source of humic acid. In an embodiment, leonardite may comprises up to 90 wt. % humic acid, by weight of the leonardite.

In an embodiment, the humus material comprises humic acid. Humic acid is produced by biodegradation of dead organic matter and represents one of the major organic compound constituents of soil (humus), peat, coal, and may constitute as much as about 95 wt. % of the total dissolved organic matter in aquatic systems. Humic acid is one of two classes of natural acidic organic polymers that are found in soil, and comprises a complex mixture of many different acids containing carboxyl and phenolate groups. In an embodiment, the humic acid comprises a compound characterized by Structure I. Humic acid can generally be characterized by a molecular weight in the range of from about 10,000 Da to about 100,000 Da.

In an embodiment, the humus material comprises fulvic acid. Fulvic acid is the other one of two classes of natural acidic organic polymers that are found in soil (humus), along with humic acid. Fulvic acid is characterized by an oxygen content about twice as high as the oxygen content of humic acid, and by a molecular weight lower than the molecular weight of the humic acid. Fulvic acid can generally be characterized by a molecular weight in the range of from about 1,000 Da to about 10,000 Da.

In an embodiment, the humus material comprises humin. Humin or humin complexes are another major constituent of soil (humus) along with humic acid and fulvic acid. Humin or humin complexes are very large substances and are considered macro-organic substances due to their molecular weights that are generally in the range of from about 100,000 Da to about 10,000,000 Da.

In an embodiment, the humus material comprises peat. Peat or turf is an accumulation of a spongy material formed by the partial decomposition of organic matter, primarily plant material, e.g., partially decayed vegetation. Peat generally forms in wetland conditions, where flooding obstructs flows of oxygen from the atmosphere, slowing rates of decomposition.

In an embodiment, the humus material comprises lignin. Lignin is a complex oxygen-containing biopolymer most commonly derived from wood. Lignin is the second most abundant organic polymer on the planet, exceeded only by cellulose.

In an embodiment, the humus material may be subjected to a dehydration process (e.g., a water or moisture removal process) prior to adding the humus material to the reaction mixture or to any pre-mixed components thereof. The dehydration of the humus materials may be accomplished by using any suitable methodology, such as for example contacting the humus materials with superheated steam, convection drying, azeotropic distillation, azeotropic distillation with xylene, toluene, benzene, mesitylene, etc. In an embodiment, the humus materials may be dehydrated by heating the humus material (for example, in an oven or dryer such as a rotary dryer) at temperatures of from about 50° C. to about 125° C., alternatively from about 55° C. to about 120° C., or alternatively from about 60° C. to about 110° C. In an embodiment, the humus material suitable for adding to the reaction mixture or to any pre-mixed components thereof comprises a water content of less than about 3.5 wt. %, alternatively less than about 3 wt. %, alternatively less than about 2.5 wt. %, or alternatively less than about 2 wt. %, by weight of the humus material. As will be appreciated by one of skill in the art, and with the help of this disclosure, the dehydration process of the humus material is meant to remove all readily removable water, such that the catalyst would not be inactivated by reacting with water. As will be appreciated by one of skill in the art, and with the help of this disclosure, while it may be desirable to remove all water from the humus material, for practical purposes it may be sufficient to remove water from the humus material down to “tightly-bound water” (e.g., hydration water) level, which tightly-bound water would not be readily available to interact with and inactivate/kill the catalyst.

In an embodiment, the humus material comprises a particle size such that equal to or greater than about 97 wt. % passes through an about 80 mesh screen (U.S. Sieve Series) and equal to or greater than about 55 wt. % passes through an about 200 mesh screen (U.S. Sieve Series); or alternatively equal to or greater than about 70 wt. % passes through an about 140 mesh screen (U.S. Sieve Series) and equal to or greater than about 60 wt. % passes through an about 170 mesh screen (U.S. Sieve Series).

A commercial example of a humus material suitable for use in the present disclosure includes CARBONOX filtration control agent. CARBONOX filtration control agent is a naturally occurring product that displays dispersive/thinning characteristics in water-based drilling fluid systems and is available from Halliburton Energy Services, Inc.

In an embodiment, the humus material is present within the reaction mixture in an amount of from about 1 wt. % to about 50 wt. %, alternatively from about 2 wt. % to about 10 wt. %, alternatively from about 3 wt. % to about 7 wt. %, or alternatively from about 3 wt. % to about 5 wt. %, based on the total weight of the reaction mixture.

In an embodiment, the reaction mixture comprises a C3+ cyclic ether. A C3+ cyclic ether refers to a cyclic ether (e.g., an epoxide or a cyclic ether with three ring atoms, generally two carbon ring atoms and one oxygen ring atom; a cyclic ether with four ring atoms, generally three carbon ring atoms and one oxygen ring atom; etc.) that has a total number of carbon atoms of equal to or greater than 3 carbon atoms, alternatively equal to or greater than 4 carbon atoms, alternatively equal to or greater than 5 carbon atoms, alternatively from about 3 carbon atoms to about 20 carbon atoms, alternatively from about 4 carbon atoms to about 15 carbon atoms, or alternatively from about 5 carbon atoms to about 10 carbon atoms. The C3+ cyclic ether may react with the humus material in the reaction mixture to yield a CAHM. Without wishing to be limited by theory, the C3+ cyclic ether may react with one or more functional groups of the humus materials, such as for example alcohol groups, phenol groups, carboxyl groups, amine groups, sulfhydryl groups, to form the CAHM. The C3+ cyclic ether may act as an alkoxylation agent in an alkoxylation reaction, e.g., the C3+ cyclic ether may alkoxylate the humus material or introduce alkoxylating elements/groups/branches in the structure of the humus material to yield a CAHM. For purposes of the disclosure herein, a single alkoxylating agent (e.g., a C3+ cyclic ether, a C3+ epoxide, oxetane, etc.) molecule that attaches to a humus material will be referred to herein as an “alkoxy unit” (e.g., a “C3+ cyclic ether unit,” a “C3+ epoxide unit,” an “oxetane unit,” etc.). In an embodiment, an alkoxylating element comprises one or more alkoxy units, which may be the same or different from each other.

In an embodiment, the C3+ cyclic ether comprises oxetane as characterized by Structure II, an epoxide (e.g., C3+ epoxide) compound characterized by Structure III, or combinations thereof,

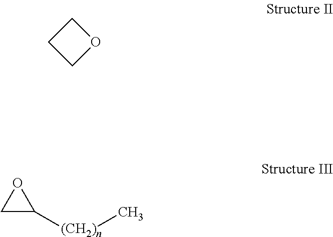

where the repeating methylene (—CH₂—) unit may occur n times with the value of n ranging from about 0 to about 3, alternatively from about 0 to about 2, or alternatively from about 0 to about 1.

In an embodiment, the C3+ cyclic ether (e.g., C3+ epoxide) characterized by Structure III comprises propylene oxide as characterized by Structure IV, butylene oxide as characterized by Structure V, pentylene oxide as characterized by Structure VI, or combinations thereof.

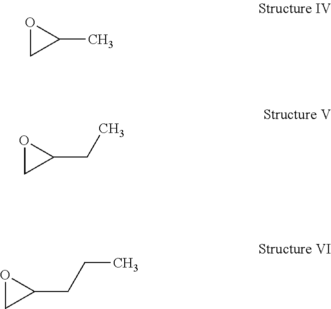

In an embodiment, the C3+ cyclic ether is present within the reaction mixture in a weight ratio of C3+ cyclic ether to humus material of from about 0.5:1 to about 50:1, alternatively from about 5:1 to about 40:1, or alternatively from about 10:1 to about 30:1.

In an embodiment, the reaction mixture comprises a catalyst. The catalyst may assist in the reaction between the humus material and the C3+ cyclic ether, but it is expected that the catalyst is not consumed during the chemical reaction (e.g., the alkoxylation of humus materials).

In an embodiment, the catalyst comprises a strong base catalyst. In an alternative embodiment, the catalyst comprises a strong acid catalyst.

Nonlimiting examples of strong base catalysts suitable for use in the present disclosure include sodium methoxide, potassium methoxide, sodium ethoxide, potassium ethoxide, and the like, or combinations thereof.

In an embodiment, the strong base catalyst is present within the reaction mixture in an amount of from about 0.1 wt. % to about 75 wt. %, alternatively from about 0.5 wt. % to about 60 wt. %, or alternatively from about 1 wt. % to about 55 wt. %, based on the total weight of the humus material.

In an embodiment, the strong acid catalyst comprises a mixture of (i) esters of titanic and/or zirconic acid with monoalkanols and (ii) sulfuric acid and/or alkanesulfonic acids and/or aryloxysulfonic acids, wherein the monoalkanols comprise from about 1 to about 4 carbon atoms, and the alkanesulfonic acids comprise from about 1 to about 6 carbon atoms. Nonlimiting examples of alkanesulfonic acids suitable for use in the present disclosure include methanesulfonic acid, ethanesulfonic acid, propanesulfonic acid, butanesulfonic acid, hexanesulfonic acids, or combinations thereof. Nonlimiting examples of aryloxysulfonic acids suitable for use in the present disclosure include phenolsulfonic acid.

In an embodiment, the strong acid catalyst comprises a mixture of (i) HF and (ii) a metal alkoxide and/or a mixed metal alkoxide, such as for example aluminum and titanium metal alkoxides and/or mixed alkoxides. In such embodiment, the metal alkoxides may be characterized by the general formula M(OCₐH₂ₐ₊₁)_{b}, wherein M is a metal, b is the valence of the metal M, and each a can independently be from about 1 to about 22 carbon atoms, alternatively from about 1 to about 18 carbon atoms, or alternatively from about 1 to about 14 carbon atoms. In an embodiment, the metal may be selected from the group consisting of aluminum, gallium, indium, thallium, titanium, zirconium and hafnium. In an embodiment, b may be either 3 or 4, depending on the valence of the metal M.

Nonlimiting examples of strong acid catalysts suitable for use in the present disclosure include HF/(CH₃O)₃Al; HF/(C₂H₅O)₃Al; HF/(CH₃O)₂(C₂H₅O)Al; HF/(C₂H₅O)₃Al; HF/(CH₃O)₂(C₂H₅O)₂Ti; HF/(CH₃O)(C₂H₅O)₃Ti; HF/(C₂₀H₄₁O)₄Ti; HF/(C₂₀H₄₁O)₃Al; HF/(i-C₃H₇O)₃Al; HF/(CH₃O)₄Ti; HF/(C₂H₅O)₄Ti; HF/(i-C₃H₇O)₄Ti; HF/(CH₃O)₄Zr; HF/(C₂H₅O)_{d}Zr, HF/(CH₃O)(C₂H₅O)(i-C₃H₇O)Al; HF/(CH₃O)₂(C₂H₅O)(i-C₃H₇O)Ti; or combinations thereof.

In an embodiment, the strong acid catalyst is present within the reaction mixture in an amount of from about 0.01 wt. % to about 10 wt. %, alternatively from about 0.05 wt. % to about 10 wt. %, or alternatively from about 0.1 wt. % to about 2 wt. %, based on the total weight of the hummus material.

In an embodiment, the reaction mixture comprises an inert reaction solvent, alternatively referred to as an inert diluent. The inert reaction solvent will not react with the catalyst (e.g., will not cause the hydrolysis of the strong base catalyst) and will also not participate in the alkoxylation reaction between the humus material and the C3+ cyclic ether, so as to avoid competing side reactions. The inert reaction solvent will not react with any of the reactants (e.g., the humus material, the C3+ cyclic ether). The inert reaction solvent will not engage in deleterious side reactions which would hinder the reaction between the humus material and the C3+ cyclic ether. Without wishing to be limited by theory, the inert reaction solvent provides a liquid medium for the alkoxylation reaction of humus materials, e.g., a liquid medium in which the reactants (e.g., the humus material, the C3+ cyclic ether) can interact and react. In an embodiment, removal of water and/or dissolved O₂ may improve the yield of the alkoxylation reaction.

In an embodiment, the inert reaction solvent may be subject to a dehydration step (e.g., the removal of water), which may be accomplished by using any suitable methodology, such as for example the use of zeolites, azeotropic distillation, pervaporation, and the like, or combinations thereof. In an embodiment, the inert reaction solvent does not comprise a substantial amount of water. In an embodiment, the reaction solvent comprises water in an amount of less than about 1 vol. %, alternatively less than about 0.1 vol. %, alternatively less than about 0.01 vol. %, alternatively less than about 0.001 vol. %, alternatively less than about 0.0001 vol. %, or alternatively less than about 0.00001 vol. %, based on the total volume of the inert reaction solvent.

In an embodiment, the inert reaction solvent may be subject to a deoxygenation step (e.g., removal of dissolved O₂), which may be accomplished by using any suitable methodology, such as for example purging an inert gas (e.g., nitrogen, helium, argon, etc.) through the inert reaction solvent (e.g., bubbling an inert gas through the solvent). In an embodiment, the inert reaction solvent does not comprise a substantial amount of dissolved O₂. In an embodiment, the reaction solvent comprises dissolved O₂ in an amount of less than about 1 wt. %, alternatively less than about 0.1 wt. %, alternatively less than about 0.01 wt. %, alternatively less than about 0.001 wt. %, alternatively less than about 0.0001 wt. %, or alternatively less than about 0.00001 wt. %, based on the total weight of the inert reaction solvent.

Nonlimiting examples of inert reaction solvents suitable for use in the present disclosure include C₆-C₁₂ liquid aromatic hydrocarbons; toluene, ethylbenzene, xylenes, o-xylene, m-xylene, p-xylene, trimethylbenzenes, cumene (i.e., isopropylbenzene), mesitylene (i.e., 1,3,5-trimethylbenzene), 1,2,4-trimethylbenzene, 1,2,3-trimethylbenzene; and the like, or combinations thereof.

As will be appreciated by one of ordinary skill in the art, and with the help of this disclosure, the term “solvent” as used herein does not imply that any or all of the reactants are solubilized in the inert reaction solvent. In an embodiment, the humus material and the catalyst are less than about 25 wt. % soluble in the inert reaction solvent, alternatively less than about 20 wt. %, alternatively less than about 15 wt. %, alternatively less than about 10 wt. %, alternatively less than about 5 wt. %, alternatively less than about 4 wt. %, alternatively less than about 3 wt. %, alternatively less than about 2 wt. %, alternatively less than about 1 wt. %, based on the weight of the inert reaction solvent. In an embodiment, the reaction mixture comprises a slurry comprising the humus material, the C3+ cyclic ether, the strong base catalyst and the inert reaction solvent. In another embodiment, the strong acid catalyst may be soluble in the inert reaction solvent. In yet another embodiment, the reaction mixture comprises a slurry comprising the humus material, the C3+ cyclic ether, the strong acid catalyst and the inert reaction solvent.

In an embodiment, the inert reaction solvent is present within the reaction mixture in an amount of from about 30 wt. % to about 90 wt. %, alternatively from about 30 wt. % to about 70 wt. %, alternatively from about 35 wt. % to about 65 wt. %, alternatively from about 40 wt. % to about 60 wt. %, or alternatively from about 45 wt. % to about 55 wt. %, based on the total weight of the reaction mixture. Alternatively, the inert reaction solvent may comprise the balance of the reaction mixture after considering the amount of the other components used.

In an embodiment, the reaction mixture optionally comprises ethylene oxide. Ethylene oxide may be used in combination with any of the C3+ cyclic ethers disclosed herein for the alkoxylation of humus materials, e.g., mixed alkoxylation of humus materials. For purposes of the disclosure herein, a single ethylene oxide molecule that attaches to a humus material will be referred to herein as an “ethoxy unit.” In an embodiment, the weight ratio between ethylene oxide and C3+ cyclic ether may be in the range of from about 10:1 to about 1:10, alternatively from about 5:1 to about 1:10, alternatively from about 5:1 to about 1:1, alternatively from about 1.5:1 to about 1:1, alternatively from about 1:1 to about 1:5, or alternatively from about 1:1 to about 1:2. When ethylene oxide is present in the reaction mixture along with the C3+ cyclic ether, the resulting CAHM recovered at the end of the reaction may be a mixed alkoxylated CAHM, such as for example a propoxylated/ethoxylated humus material, a butoxylated/ethoxylated humus material, a pentoxylated/ethoxylated humus material, etc.

In an embodiment, the C3+ alkoxylated humus materials (CAHMs) may be produced by heating a reaction mixture comprising a humus material, a C3+ cyclic ether, a catalyst and an inert reaction solvent. In an embodiment, the reaction mixture may be heated by using any suitable methodology (e.g., a fired heater, heat exchanger, heating mantle, burners, etc.) to a temperature ranging from about 130° C. to about 170° C., alternatively from about 140° C. to about 160° C., or alternatively from about 145° C. to about 155° C. In an embodiment, the reaction mixture may be heated to a temperature of about 150° C.

In an embodiment, the reaction mixture may be heated (e.g., reacted) in a substantially oxygen-free atmosphere. For purposes of the disclosure herein, the term “atmosphere” refers to any space within the reaction vessel that is not occupied by the reaction mixture or any parts of the reaction vessel (e.g., a stirring device), for example a head space within a reactor vessel. In an embodiment, a substantially oxygen-free atmosphere comprises oxygen in an amount of less than about 1 vol. %, alternatively less than about 0.1 vol. %, alternatively less than about 0.01 vol. %, alternatively less than about 0.001 vol. %, alternatively less than about 0.0001 vol. %, or alternatively less than about 0.00001 vol. %, based on the total volume of the atmosphere in which the alkoxylation of the humus materials is carried out.

In an embodiment, the substantially oxygen-free atmosphere may be obtained by using any suitable methodology, such as for example purging a reaction vessel comprising the reaction mixture or any components thereof with an inert gas, i.e., a gas that does not participate in the alkoxylation reaction. For example, the reaction mixture may be maintained under an inert gas blanket for the duration of the alkoxylation reaction. Nonlimiting examples of inert gases suitable for use in the present disclosure include nitrogen, helium, argon, or combinations thereof.

In an embodiment, the components of the reaction mixture (e.g., the humus material, the C3+ cyclic ether, the catalyst and the inert reaction solvent) may be heated while being mixed together, and the heating may continue for the duration of the chemical modification reaction (e.g., alkoxylation of humus materials). In another embodiment, all components of the reaction mixture (e.g., the humus material, the C3+ cyclic ether, the catalyst and the inert reaction solvent) may be mixed together to form the reaction mixture prior to heating the reaction mixture. In an alternative embodiment, at least two components of the reaction mixture are pre-mixed and heated prior to the addition of the other components. In some embodiments, the humus material, the C3+ cyclic ether, and the catalyst may each be pre-mixed individually with a portion of the inert reaction solvent and heated, and then they may be mixed together in any suitable sequence to form the reaction mixture. In an embodiment, the mixing or pre-mixing of any of the components of the reaction mixture (e.g., the humus material, the C3+ cyclic ether, the catalyst and the inert reaction solvent) may be carried out under stirring or agitation by using any suitable methodology (e.g., magnetic stirring, mechanical stirring, rotated reaction vessel having internal mixing structures, etc.). In an embodiment, the humus material, the catalyst and the inert reaction solvent are pre-mixed and heated prior to the addition of the C3+ cyclic ether to form the reaction mixture. When any of the components of the reaction mixture are pre-mixed, such pre-mixing generally occurs at the temperature at which it is intended to carry out the chemical modification of the humus materials (e.g., alkoxylation of humus materials), e.g., a temperature ranging from about 130° C. to about 170° C. In an embodiment, when a component of the reaction mixture is added to pre-mixed components, such addition may occur by adding all at once the entire amount of the component to the pre-mixed components. In an alternative embodiment, the component may be added in different portions/aliquots/charges to the pre-mixed components over a desired time period. For example, the total amount of the C3+ cyclic ether may be divided into a plurality of portions, which may have either have equal weights or have weights different from each other, and each portion of the C3+ cyclic ether may be added to the pre-mixed components (e.g., the pre-mixed humus material, catalyst and inert reaction solvent) over a desired time period, such as for example each portion of C3+ cyclic ether may be added to the pre-mixed components every hour. In an embodiment, when the C3+ cyclic ether is added to the other pre-mixed components in portions, the conditions (e.g., temperature, pressure) inside the reaction vessel where the chemical modification of the humus materials (e.g., alkoxylation of humus materials) is carried out might vary while each C3+ cyclic ether portion reacts with the humus material (e.g., alkoxylates the humus material). In such embodiment, the following portion of the C3+ cyclic ether may be added to the reaction vessel after the conditions (e.g., temperature, pressure) inside the reaction vessel have equilibrated (e.g., have reached a steady state, which may be the same or different when compared to the steady state conditions inside the reaction vessel prior to the addition of the previous portion of the C3+ cyclic ether).

In an embodiment, the reaction mixture or any pre-mixed components thereof may be heated in a substantially oxygen-free atmosphere to carry out the chemical modification of the humus materials, e.g., alkoxylation of humus materials. In an embodiment, the components of the reaction mixture (e.g., the humus material, the C3+ cyclic ether, the catalyst and the inert reaction solvent) may be mixed or pre-mixed in a substantially oxygen-free atmosphere. In an embodiment, the humus material, the catalyst and the inert reaction solvent are pre-mixed and heated in a substantially oxygen-free atmosphere prior to the addition of the C3+ cyclic ether.

In an embodiment, the components of the reaction mixture (e.g., the humus material, the C3+ cyclic ether, the catalyst and the inert reaction solvent) may be mixed or pre-mixed as previously described herein at a pressure at which it is intended to carry out the chemical modification reaction (e.g., alkoxylation of humus materials), e.g., a pressure in the range of from about 32 psi to about 300 psi, alternatively from about 25 psi to 250 psi, or alternatively from about 20 psi to 200 psi.

In an embodiment, the chemical modification reaction (e.g., alkoxylation of humus materials) may be carried out over a time period ranging from about 0.5 h to about 10 h, alternatively from about 0.5 h to about 7 h, or alternatively from about 0.5 h to about 3 h. In an embodiment, when any of the components of the reaction mixture (e.g., the humus material, the C3+ cyclic ether, the catalyst and the inert reaction solvent) are pre-mixed, such pre-mixing may occur for a time period ranging from about 0.5 h to about 1.5 h, or alternatively from about 0.5 h to about 1 h.

In an embodiment, the CAHM may be recovered from the reaction mixture at the end of the alkoxylation reaction. The reaction may be terminated by removing the heat source and returning (e.g., cooling down) the reaction mixture to a temperature lower than the temperature required for the alkoxylation reaction, e.g., a temperature lower than about 130° C. The reaction mixture may be filtered to remove any solid particulates that might still be present in the reaction mixture.

In an embodiment, the inert reaction solvent may be removed from the reaction mixture at the end of the alkoxylation reaction by using any suitable methodology, such as for example flash evaporation, distillation, liquid-liquid-extraction, or combinations thereof. The removal of the inert reaction solvent may generally yield the CAHMs (e.g., recovered CAHMs). Depending on the degree of alkoxylation of the CAHMs (e.g., the extent of the chemical modification of the humus materials), the state of matter of the recovered CAHMs may range from a liquid to a solid. As will be appreciated by one of ordinary skill in the art, and with the help of this disclosure, the degree of alkoxylation of the CAHMs (e.g., the extent of the chemical modification of the humus materials) is dependent on the ratio of the C3+ cyclic ether to the humus material in the reaction mixture.

In an embodiment, the CAHMs may be a liquid when the weight ratio of C3+ cyclic ether to humus material ranges from about 2:1 to about 15:1. In another embodiment, the CAHMs may be a greasy wax when the weight ratio of C3+ cyclic ether to humus material is from about 15:1 to about 20:1. In yet another embodiment, the CAHMs may be a waxy solid when the weight ratio of C3+ cyclic ether to humus material is from about 20:1 to about 30:1. In still yet another embodiment, the CAHMs may be a solid when the weight ratio of C3+ cyclic ether to humus material ranges from about 30:1 to about 50:1. Generally, the CAHMs may be soluble in polar solvents such as water and methanol and insoluble in alkanes, hexane, pentane, and the like. Without wishing to be limited by theory, the higher the degree of alkoxylation of the CAHMs (e.g., the extent of the chemical modification of the humus materials), the higher the solubility of the CAHMs in polar solvents. The CAHMs may also be soluble to some extent (e.g., slightly soluble) in aromatic hydrocarbons, and temperatures above the ambient temperature increase the solubility of CAHMs in aromatic hydrocarbons. In an embodiment, the liquid CAHMs may be slightly soluble in water and xylene. In an embodiment, the greasy wax CAHMs may be slightly soluble in dimethyl formamide, and soluble in water and xylene. In an embodiment, the waxy solid CAHMs may be soluble in dimethyl formamide and xylene, and very soluble in water. In an embodiment, the solid CAHMs may be very soluble in dimethyl formamide, xylene, and water. For the purposes of the disclosure herein, “insoluble” refers to a solubility of less than 1.0 g/L in a particular solvent; “slightly soluble” refers to a solubility of from about 1.0 g/L to about 2.0 g/L in a particular solvent; “soluble” refers to a solubility of from about 2.0 g/L to about 20.0 g/L in a particular solvent; and “very soluble” refers to a solubility of equal to or greater than about 20.0 g/L in a particular solvent; wherein all solubility values are given at room temperature, unless otherwise noted.

In an embodiment, the CAHM obtained as previously described herein by using a strong base catalyst comprises a compound characterized by Structure VII:

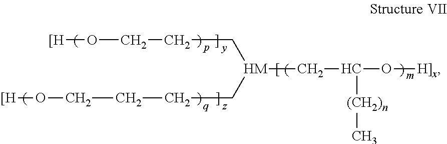

where HM represents the humus material; the repeating methylene (—CH₂—) unit may occur n times with the value of n ranging from about 0 to about 3, alternatively from about 0 to about 2, or alternatively from about 0 to about 1, as previously described for the C3+ cyclic ether (e.g., C3+ epoxide) compound characterized by Structure III; a repeating C3+ cyclic ether unit or C3+ epoxide unit that originates from the C3+ cyclic ether (e.g., C3+ epoxide) in the presence of a strong base catalyst may occur m times with the value of m ranging from about 1 to about 30, alternatively from about 2 to about 20, or alternatively from about 2 to about 10; a C3+ alkoxylating element may occur x times with the value of x ranging from about 0 to about 300, alternatively from about 2 to about 250, or alternatively from about 10 to about 200, per 100 g of humus material; a repeating ethoxy unit (e.g., when the optional ethylene oxide is used in the alkoxylation along with the C3+ cyclic ether) may occur p times with the value of p ranging from about 1 to about 30, alternatively from about 2 to about 20, or alternatively from about 2 to about 10; an ethoxylating element may occur y times with the value of y ranging from about 0 to about 200, alternatively from about 1 to about 150, or alternatively from about 2 to about 100, per 100 g of humus material; a repeating oxetane unit (e.g., when the C3+ cyclic ether used in the alkoxylation comprises oxetane as characterized by Structure II) may occur q times with the value of q ranging from about 1 to about 30, alternatively from about 2 to about 20, or alternatively from about 2 to about 10; and a C3+ alkoxylating element may occur z times with the value of z ranging from about 0 to about 300, alternatively from about 1 to about 250, or alternatively from about 2 to about 200, per 100 g of humus material. As will be appreciated by one of skill in the art, and with the help of this disclosure, x and z cannot both be 0 at the same time. For purposes of the disclosure herein, one or more alkoxy or alkoxylating units (e.g., a C3+ cyclic ether unit, an oxetane unit, an ethoxy unit) that attach to the humus material structure in the same point (e.g., via the same functional group of the humus material) will be referred to herein as an “alkoxyating element” (e.g., “C3+ alkoxylating element,” “ethoxylating element”). The C3+ alkoxylating element refers to an alkoxyating element that originates from a C3+ cyclic ether, such as for example oxetane, a C3+ epoxide, etc. For purposes of the disclosure herein, the description of various substituents (e.g., a substituent of a CAHM, such as for example a C3+ alkoxylating element, an ethoxylating element, etc.) and parameters thereof (e.g., x, x1, y, z, p, q, m, m1) is understood to apply to all related structures, unless otherwise designated herein.

In an embodiment, the CAHM obtained as previously described herein by using a strong acid catalyst comprises a compound characterized by Structure VIII:

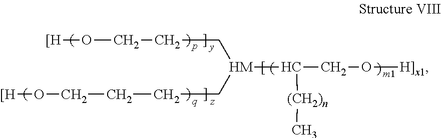

where the repeating C3+ cyclic ether unit that originates from the C3+ cyclic ether in the presence of a strong acid catalyst may occur m1 times with the value of m1 ranging from about 1 to about 30, alternatively from about 2 to about 20, or alternatively from about 2 to about 10; and the C3+ alkoxylating element may occur x1 times with the value of x1 ranging from about 0 to about 300, alternatively from about 2 to about 250, or alternatively from about 10 to about 200, per 100 g of humus material. As will be appreciated by one of skill in the art, and with the help of this disclosure, x1 and z cannot both be 0 at the same time.

Without wishing to be limited by theory, the functional groups of the humus material may act as the nucleophile in the alkoxylation reaction in the presence of a strong base, thereby attacking the C3+ cyclic ether ring (e.g., the cyclic ether ring of the compound characterized by Structure III) at the least substituted carbon atom. Further, without wishing to be limited by theory, it is expected that the alkoxylation reaction between the humus material and the C3+ cyclic ether in the presence of a strong base will yield the compound characterized by Structure VII, due both to the presence of the strong base catalyst and to major steric hinderance between the very bulky humus material and the alkyl chain (e.g., (CH₂)ₙCH₃) present in the C3+ cyclic ether compound characterized by Structure III. While unlikely, it might be possible that a small amount of a compound characterized by Structure VIII would form during the alkoxylation of the humus material in the presence of a strong base.

In an embodiment, the CAHMs obtained as previously described herein by using a strong base catalyst may comprise a compound characterized by Structure VIII in an amount of less than about 10 wt. %, alternatively less than about 9 wt. %, alternatively less than about 8 wt. %, alternatively less than about 7 wt. %, alternatively less than about 6 wt. %, alternatively less than about 5 wt. %, alternatively less than about 4 wt. %, alternatively less than about 3 wt. %, alternatively less than about 2 wt. %, alternatively less than about 1 wt. %, alternatively less than about 0.1 wt. %, alternatively less than about 0.01 wt. %, alternatively less than about 0.001 wt. %, alternatively less than about 0.0001 wt. %, based on the total weight of the CAHM.

Without wishing to be limited by theory, in the presence of a strong acid catalyst, the C3+ cyclic ether ring deprotonates the strong acid, thereby creating a protonated C3+ cyclic ether ring intermediate having a positive charge that is delocalized between the O atom of the cyclic ether ring and the most substituted carbon atom adjacent to the O atom of the cyclic ether ring, thereby enabling the functional groups of the humus material to act as the nucleophile in the alkoxylation reaction, and attack the C3+ cyclic ether ring (e.g., the cyclic ether ring of the compound characterized by Structure III) at the most substituted carbon atom. Further, without wishing to be limited by theory, it is expected that the alkoxylation reaction between the humus material and the C3+ cyclic ether in the presence of a strong acid will yield the compound characterized by Structure VIII, due to the presence of the strong acid catalyst. While unlikely, it might be possible that a small amount of a compound characterized by Structure VII would form during the alkoxylation of the humus material in the presence of a strong acid.

In an embodiment, the CAHMs obtained as previously described herein by using a strong acid catalyst may comprise a compound characterized by Structure VII in an amount of less than about 10 wt. %, alternatively less than about 9 wt. %, alternatively less than about 8 wt. %, alternatively less than about 7 wt. %, alternatively less than about 6 wt. %, alternatively less than about 5 wt. %, alternatively less than about 4 wt. %, alternatively less than about 3 wt. %, alternatively less than about 2 wt. %, alternatively less than about 1 wt. %, alternatively less than about 0.1 wt. %, alternatively less than about 0.01 wt. %, alternatively less than about 0.001 wt. %, alternatively less than about 0.0001 wt. %, based on the total weight of the CAHM.

As will be appreciated by one of skill in the art, and with the help of this disclosure, a CAHMs obtained by using a strong acid catalyst may be combined with a CAHM obtained by using a strong base catalyst, as it may be desirable to modulate the properties (e.g., solubility, melting point, thermal stability, etc.) of the CAHM to be used in further applications.

In an embodiment, the CAHM comprises a multi-branched structure, wherein each branch comprises repeating alkoxy units, such as for example repeating C3+ cyclic ether units (e.g., C3+ epoxide unit, oxetane unit) and/or repeating ethoxy units, as shown in Structure VII and/or Structure VIII. For example, each branch of the CAHM is represented in Structure VII by each of the x C3+ alkoxylating elements, by each of the y ethoxylating elements, or by each of the z C3+ alkoxylating elements. For example, each branch of the CAHM is represented in Structure VIII by each of the x1 C3+ alkoxylating elements, by each of the y ethoxylating elements, or by each of the z C3+ alkoxylating elements. In an embodiment, the branch of a CAHM may comprise a C3+ alkoxylating element of Structure VII, an ethoxylating element, or combinations thereof. In an embodiment, the branch of a CAHM may comprise a C3+ alkoxylating element of Structure VIII, an ethoxylating element, or combinations thereof.

In an embodiment, a CAHM obtained by using a strong base catalyst may comprise a repeating C3+ cyclic ether unit (e.g., C3+ epoxide unit) as shown in Structure VIII in an amount of less than about 10 wt. %, alternatively less than about 9 wt. %, alternatively less than about 8 wt. %, alternatively less than about 7 wt. %, alternatively less than about 6 wt. %, alternatively less than about 5 wt. %, alternatively less than about 4 wt. %, alternatively less than about 3 wt. %, alternatively less than about 2 wt. %, alternatively less than about 1 wt. %, alternatively less than about 0.1 wt. %, alternatively less than about 0.01 wt. %, alternatively less than about 0.001 wt. %, alternatively less than about 0.0001 wt. %, based on the total weight of the CAHM obtained by using a strong base catalyst.

In an embodiment, a CAHM obtained by using a strong acid catalyst may comprise a repeating C3+ cyclic ether unit (e.g., C3+ epoxide unit) as shown in Structure VII in an amount of less than about 10 wt. %, alternatively less than about 9 wt. %, alternatively less than about 8 wt. %, alternatively less than about 7 wt. %, alternatively less than about 6 wt. %, alternatively less than about 5 wt. %, alternatively less than about 4 wt. %, alternatively less than about 3 wt. %, alternatively less than about 2 wt. %, alternatively less than about 1 wt. %, alternatively less than about 0.1 wt. %, alternatively less than about 0.01 wt. %, alternatively less than about 0.001 wt. %, alternatively less than about 0.0001 wt. %, based on the total weight of the CAHM obtained by using a strong acid catalyst.

As will be apparent to one of skill in the art, and with the help of this disclosure, each of the x C3+ alkoxylating elements and/or C3+ alkoxylating branches of Structure VII may independently comprise lengths (e.g., numbers (m) of cyclic ether units) that may be the same or different when compared to the lengths (e.g., numbers (m) of cyclic ether units) of the other C3+ alkoxylating elements (e.g., C3+ alkoxylating branches). For example, one or more of the C3+ alkoxylating elements (e.g., C3+ alkoxylating branches) of Structure VII may comprise m=5 C3+ cyclic ether units; one or more of the C3+ alkoxylating elements (e.g., C3+ alkoxylating branches) may comprise m=4 C3+ cyclic ether units; one or more of the C3+ alkoxylating elements (e.g., C3+ alkoxylating branches) may comprise m=8 C3+ cyclic ether units; etc. Similarly, when oxetane as characterized by Structure II is used in the alkoxylation reaction, each of the z C3+ alkoxylating elements and/or C3+ alkoxylating branches of Structure VII and/or Structure VIII may independently comprise lengths (e.g., numbers (q) of oxetane units) that may be the same or different when compared to the lengths (e.g., numbers (q) of oxetane units) of the other C3+ alkoxylating elements (e.g., C3+ alkoxylating branches). For example, one or more of the z C3+ alkoxylating elements (e.g., C3+ alkoxylating branches) of Structure VII and/or Structure VIII may comprise q=5 oxetane units; one or more of the z C3+ alkoxylating elements (e.g., C3+ alkoxylating branches) may comprise q=4 oxetane units; one or more of the z C3+ alkoxylating elements (e.g., C3+ alkoxylating branches) may comprise q=8 oxetane units; etc. Similarly, when the optional ethylene oxide is used in the alkoxylation reaction along with the C3+ cyclic ether (e.g., y≠0), each of the y ethoxylating elements and/or ethoxylating branches of Structure VII and/or Structure VIII may independently comprise lengths (e.g., numbers (p) of ethoxy units) that may be the same or different when compared to the lengths (e.g., numbers (p) of ethoxy units) of the other ethoxylating elements (e.g., ethoxylating branches). For example, one or more of the ethoxylating elements (e.g., ethoxylating branches) of Structure VII and/or Structure VIII may comprise p=5 ethoxy units; one or more of the ethoxylating elements (e.g., ethoxylating branches) may comprise p=4 ethoxy units; one or more of the ethoxylating elements (e.g., ethoxylating branches) may comprise p=8 ethoxy units; etc.

As will be apparent to one of ordinary skill in the art, and with the help of this disclosure, more than one type of C3+ cyclic ether may be used in the same alkoxylation reaction of the humus material, and as such one or more of the x C3+ alkoxylating elements (e.g., C3+ alkoxylating branches) of Structure VII and/or one or more of the x1 C3+ alkoxylating elements (e.g., C3+ alkoxylating branches) of Structure VIII may comprise different types of cyclic ether units (e.g., propylene oxide, butylene oxide, pentylene oxide, etc.). For example, some of the C3+ alkoxylating elements (e.g., C3+ alkoxylating branches) of Structure VII and/or Structure VIII may comprise only one type of cyclic ether unit (e.g., propylene oxide); other C3+ alkoxylating elements (e.g., C3+ alkoxylating branches) of Structure VII and/or Structure VIII may comprise only one type of a different type of cyclic ether unit (e.g., butylene oxide); other C3+ alkoxylating elements (e.g., C3+ alkoxylating branches) of Structure VII and/or Structure VIII may comprise only one type of another type of cyclic ether unit (e.g., oxetane); one or more of the C3+ alkoxylating elements (e.g., C3+ alkoxylating branches) of Structure VII and/or Structure VIII may comprise two types of cyclic ether units (e.g., propylene oxide and butylene oxide); one or more of the C3+ alkoxylating elements (e.g., C3+ alkoxylating branches) of Structure VII and/or Structure VIII may comprise three types of cyclic ether units (e.g., propylene oxide, butylene oxide, and oxetane); etc. Similarly, when the optional ethylene oxide is used in the alkoxylation reaction along with the C3+ cyclic ether (e.g., y≠0), each of the alkoxylating elements (e.g., alkoxylating branches) of Structure VII and/or Structure VIII (e.g., C3+ alkoxylating element, ethoxylating element) may independently comprise both ethoxy units and C3+ cyclic ether units.

In an embodiment, when more than one type of alkoxylating agent (e.g., C3+ cyclic ether, propylene oxide, butylene oxide, pentylene oxide, oxetane, ethylene oxide, etc.) is used during the alkoxylation reaction of the humus material, all alkoxylating agents (e.g., C3+ cyclic ether, propylene oxide, butylene oxide, pentylene oxide, oxetane, ethylene oxide, etc.) may be added into the reaction vessel at the same time. In an alternative embodiment, the alkoxylating agents (e.g., C3+ cyclic ether, propylene oxide, butylene oxide, pentylene oxide, oxetane, ethylene oxide, etc.) may be added into the reaction vessel at different times. In some embodiments, the alkoxy units may form new alkoxylated elements/branches, or may extend already existing alkoxylated elements/branches. In yet other embodiments, the humus material may be alkoxylated with one type of alkoxylating agent (e.g., C3+ cyclic ether, propylene oxide, butylene oxide, pentylene oxide, oxetane, ethylene oxide, etc.) and then recovered as a first CAHM, and the first CAHM may be used as the humus material in a subsequent alkoxylation reaction with a different type of alkoxylating agent (e.g., C3+ cyclic ether, propylene oxide, butylene oxide, pentylene oxide, oxetane, ethylene oxide, etc.) and then recovered as a second CAHM. In such embodiments, the second CAHM may comprise alkoxylated elements/branches of the first CAHM, alkoxylated elements/branches that were newly formed in the subsequent alkoxylation reaction, and alkoxylated elements/branches that were formed by adding alkoxy units to the alkoxylated elements/branches of the first CAHM. As will be appreciated by one of skill in the art, and with the help of this disclosure, a CAHM produced in the presence of a strong acid catalyst may be used as the humus material in a subsequent alkoxylation reaction that may take place in the presence of a strong base catalyst. Similarly, as will be appreciated by one of skill in the art, and with the help of this disclosure, a CAHM produced in the presence of a strong base catalyst may be used as the humus material in a subsequent alkoxylation reaction that may take place in the presence of a strong acid catalyst.

In an embodiment, the structure of the compound characterized by Structure VII and/or the structure of the compound characterized by Structure VIII may be confirmed by running structure analysis tests. Nonlimiting examples of structure analysis tests suitable for use in the present disclosure include ash analysis for mineral content; elemental ash analysis; elemental analysis for C, H, O, N, S, which could also provide some information regarding the ratio of different alkoxy units in the CAHM, such as for example the ratio of propylene oxide or propoxy units to ethoxy units in the CAHM, in the case of an alkoxylation reaction where both propylene oxide and ethylene oxide are used; infrared or IR spectroscopy, which could provide information with respect to carboxylic groups differences between the humus material and the CAHM, as well as identify the presence of different alkoxy units in the CAHM, such as for example the propoxy units and ethoxy units in the CAHM; ultraviolet-visible or UV-Vis spectroscopy which could provide information regarding the presence of alkoxy units in the CAHM; nuclear magnetic resonance or NMR spectroscopy for CAHMs soluble in D₂O (i.e., deuterated water) and/or CDCl₃ (deuterated chloroform), to identify the presence of different alkoxy units in the CAHM, such as for example the propoxy units and ethoxy units in the CAHM, as well as their ratios with respect to each other; thermogravimetric analysis or TGA for investigating the CAHM profile loss of weight versus temperature, i.e., CAHM thermal stability; differential thermal analysis or DTA to record the exotherm thermograms or the endotherm thermograms; differential scanning calorimetry or DSC; gel permeation chromatography and low-angle laser light scattering to determine the MW of the CAHMs; and the like.

In an embodiment, the CAHM disclosed herein does not include ethoxylated humus materials characterized by the general formula L-(CH₂—CH₂—O)_{w}H as disclosed in U.S. Pat. No. 4,578,456, wherein L can be a humus material, lignite, and 4.55≦w≦227 per 100 g of humus material or lignite.

In an embodiment, the reaction mixture excludes ethylene oxide. In an embodiment, the reaction mixture does not contain a material amount of ethylene oxide. In an embodiment, the reaction mixture comprises ethylene oxide in an amount of less than about 1 wt. %, alternatively less than about 0.1 wt. %, alternatively less than about 0.01 wt. %, alternatively less than about 0.001 wt. %, alternatively less than about 0.0001 wt. %, alternatively less than about 0.00001 wt. %, or alternatively less than about 0.000001 wt. %, based on the total weight of the reaction mixture. In such embodiment, referring to the CAHM characterized by Structure VII and/or to the CAHM characterized by Structure VIII, y=0. In such embodiment, the CAHM characterized by Structure VII comprises a compound characterized by Structure IX, and/or the CAHM characterized by Structure VIII comprises a compound characterized by Structure X:

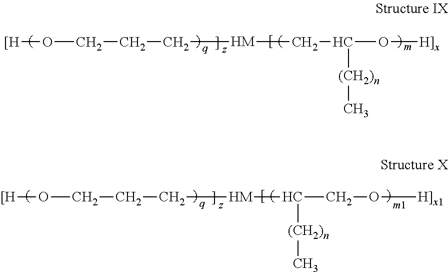

where HM represents the humus material; the repeating methylene (—CH₂—) unit may occur n times with the value of n ranging from about 0 to about 3, alternatively from about 0 to about 2, or alternatively from about 0 to about 1, as previously described for the C3+ cyclic ether compound characterized by Structure III; the repeating C3+ cyclic ether unit that originates from the C3+ cyclic ether (e.g., C3+ epoxide) in the presence of a strong base catalyst may occur m times with the value of m ranging from about 1 to about 30, alternatively from about 2 to about 20, or alternatively from about 2 to about 10; the repeating C3+ cyclic ether unit that originates from the C3+ cyclic ether (e.g., C3+ epoxide) in the presence of a strong acid catalyst may occur m1 times with the value of m1 ranging from about 1 to about 30, alternatively from about 2 to about 20, or alternatively from about 2 to about 10; the C3+ alkoxylating element may occur x times with the value of x ranging from about 0 to about 300, alternatively from about 2 to about 250, or alternatively from about 10 to about 200, per 100 g of humus material; the C3+ alkoxylating element may occur x1 times with the value of x1 ranging from about 0 to about 300, alternatively from about 2 to about 250, or alternatively from about 10 to about 200, per 100 g of humus material; the repeating oxetane unit (e.g., when the C3+ cyclic ether used in the alkoxylation comprises oxetane as characterized by Structure II) may occur q times with the value of q ranging from about 1 to about 30, alternatively from about 2 to about 20, or alternatively from about 2 to about 10; and the C3+ alkoxylating element may occur z times with the value of z ranging from about 0 to about 300, alternatively from about 1 to about 250, or alternatively from about 2 to about 200, per 100 g of humus material. As will be appreciated by one of skill in the art, and with the help of this disclosure, x and z cannot both be 0 at the same time. Similarly, as will be appreciated by one of skill in the art, and with the help of this disclosure, x1 and z cannot both be 0 at the same time.

In an embodiment, the CAHM characterized by Structure IX comprises a propoxylated humus material characterized by Structure XI, a propoxylated/butoxylated humus material characterized by Structure XII, a propoxylated/pentoxylated humus material characterized by Structure XIII, and the like, or combinations thereof. As will be appreciated by one of skill in the art, and with the help of this disclosure, the alkoxylation of a humus material with oxetane results in a propoxylated humus material. Further, as will be appreciated by one of skill in the art, and with the help of this disclosure, a propoxylated humus material may comprise oxetane units, propoxy units that originate in an alkoxylating agent comprising propylene oxide as characterized by Structure IV, or combinations thereof.

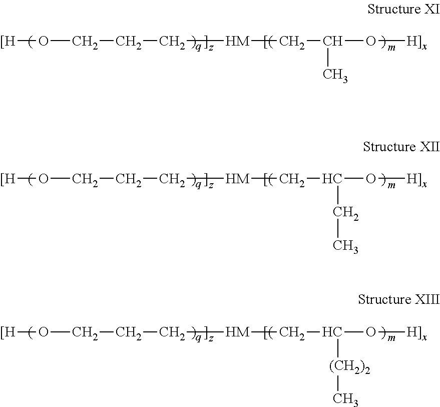

In an embodiment, the CAHM characterized by Structure X comprises a propoxylated humus material characterized by Structure XIV, a propoxylated/butoxylated humus material characterized by Structure XV, a propoxylated/pentoxylated humus material characterized by Structure XVI, and the like, or combinations thereof.

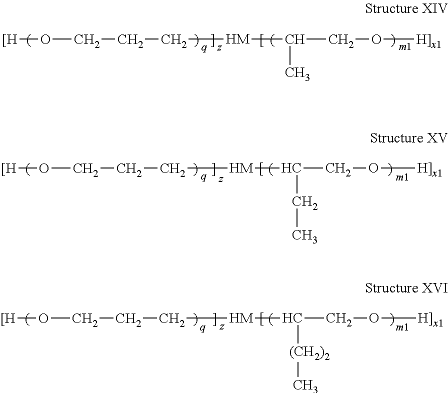

In an embodiment, the reaction mixture excluding ethylene oxide further excludes oxetane as characterized by Structure II. In such embodiment, the reaction mixture does not contain a material amount of oxetane. In such embodiment, the reaction mixture comprises oxetane in an amount of less than about 1 wt. %, alternatively less than about 0.1 wt. %, alternatively less than about 0.01 wt. %, alternatively less than about 0.001 wt. %, alternatively less than about 0.0001 wt. %, alternatively less than about 0.00001 wt. %, or alternatively less than about 0.000001 wt. %, based on the total weight of the reaction mixture. In such embodiment, referring to the CAHM characterized by Structure IX and/or to the CAHM characterized by Structure X, z=0. In such embodiment, the CAHM characterized by Structure IX comprises a compound characterized by Structure XVII, and/or the CAHM characterized by Structure X comprises a compound characterized by Structure XVIII:

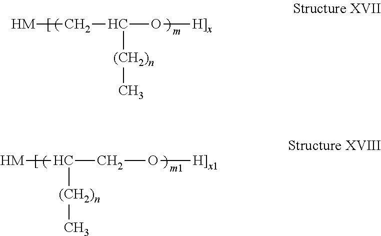

where HM represents the humus material; the repeating methylene (—CH₂—) unit may occur n times with the value of n ranging from about 0 to about 3, alternatively from about 0 to about 2, or alternatively from about 0 to about 1, as previously described for the C3+ cyclic ether compound characterized by Structure III; the repeating C3+ cyclic ether unit that originates from the C3+ cyclic ether in the presence of a strong base catalyst may occur m times with the value of m ranging from about 1 to about 30, alternatively from about 2 to about 20, or alternatively from about 2 to about 10; the repeating C3+ cyclic ether unit that originates from the C3+ cyclic ether (e.g., C3+ epoxide) in the presence of a strong acid catalyst may occur m1 times with the value of m1 ranging from about 1 to about 30, alternatively from about 2 to about 20, or alternatively from about 2 to about 10; the C3+ alkoxylating element may occur x times with the value of x ranging from about 1 to about 300, alternatively from about 2 to about 250, or alternatively from about 10 to about 200, per 100 g of humus material; the C3+ alkoxylating element may occur x1 times with the value of x1 ranging from about 1 to about 300, alternatively from about 2 to about 250, or alternatively from about 10 to about 200, per 100 g of humus material.

In an embodiment, the CAHM characterized by Structure XVII comprises a propoxylated humus material characterized by Structure XIX, a butoxylated humus material characterized by Structure XX, a pentoxylated humus material characterized by Structure XXI, and the like, or combinations thereof.

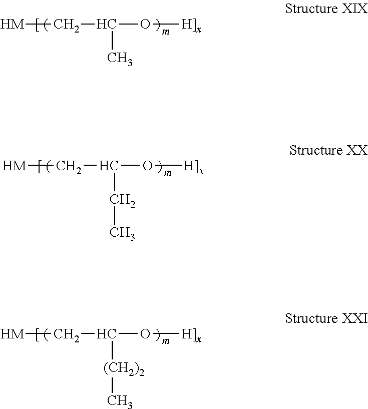

In an embodiment, the CAHM characterized by Structure XVIII comprises a propoxylated humus material characterized by Structure XXII, a butoxylated humus material characterized by Structure XXIII, a pentoxylated humus material characterized by Structure XXIV, and the like, or combinations thereof.

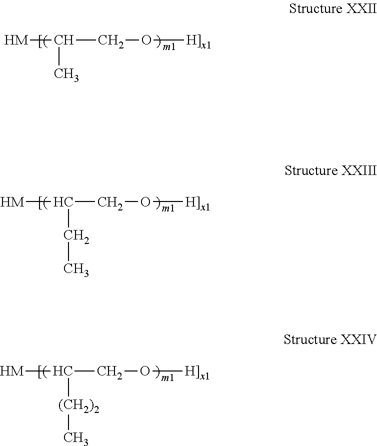

In an embodiment, the reaction mixture excluding ethylene oxide further excludes an epoxide (e.g., C3+ epoxide) compound characterized by Structure III. In such embodiment, the reaction mixture does not contain a material amount of an epoxide (e.g., C3+ epoxide) compound characterized by Structure III. In such embodiment, the reaction mixture comprises an epoxide (e.g., C3+ epoxide) compound characterized by Structure III in an amount of less than about 1 wt. %, alternatively less than about 0.1 wt. %, alternatively less than about 0.01 wt. %, alternatively less than about 0.001 wt. %, alternatively less than about 0.0001 wt. %, alternatively less than about 0.00001 wt. %, or alternatively less than about 0.000001 wt. %, based on the total weight of the reaction mixture. In such embodiment, referring to the CAHM characterized by Structure IX, x=0. In such embodiment, referring to the CAHM characterized by Structure X, x1=0. In such embodiment, the CAHM characterized by Structure IX and/or the CAHM characterized by Structure X comprise a propoxylated humus material characterized by Structure XXV:

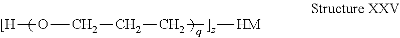

where HM represents the humus material; the repeating oxetane unit (e.g., when the C3+ cyclic ether used in the alkoxylation comprises oxetane as characterized by Structure II) may occur q times with the value of q ranging from about 1 to about 30, alternatively from about 2 to about 20, or alternatively from about 2 to about 10; and the C3+ alkoxylating element may occur z times with the value of z ranging from about 1 to about 300, alternatively from about 1 to about 250, or alternatively from about 2 to about 200, per 100 g of humus material.

In an embodiment, the reaction mixture comprises a strong base catalyst and optionally ethylene oxide along with the C3+ cyclic ether, as previously described herein. In such embodiment, referring to the CAHM characterized by Structure VII, y≠0. In such embodiment, the CAHM characterized by Structure VII comprises a propoxylated/ethoxylated humus material characterized by Structure XXVI, a butoxylated/propoxylated/ethoxylated humus material characterized by Structure XXVII, a pentoxylated/propoxylated/ethoxylated humus material characterized by Structure XXVIII, and the like, or combinations thereof.

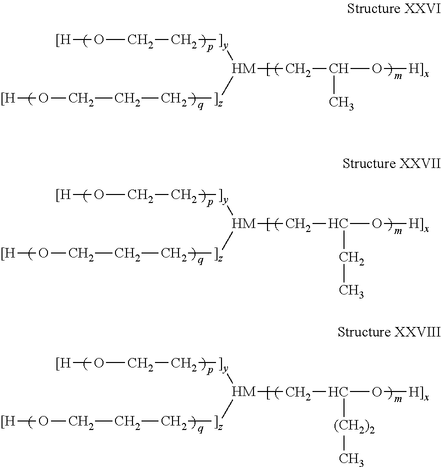

In an embodiment, the reaction mixture comprises a strong acid catalyst and optionally ethylene oxide along with the C3+ cyclic ether, as previously described herein. In such embodiment, referring to the CAHM characterized by Structure VIII, y≠0. In such embodiment, the CAHM characterized by Structure VIII comprises a propoxylated/ethoxylated humus material characterized by Structure XXIX, a butoxylated/propoxylated/ethoxylated humus material characterized by Structure XXX, a pentoxylated/propoxylated/ethoxylated humus material characterized by Structure XXXI, and the like, or combinations thereof.

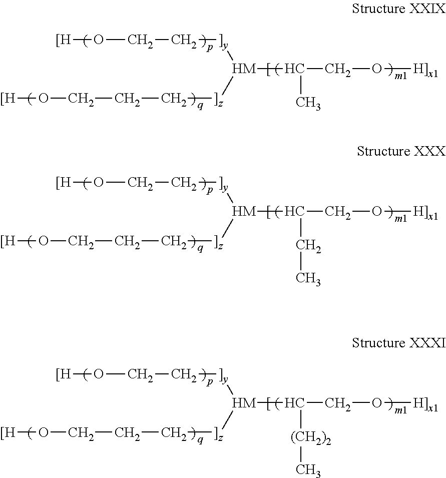

In an embodiment, the reaction mixture excludes oxetane. In an embodiment, the reaction mixture does not contain a material amount of oxetane. In an embodiment, the reaction mixture comprises oxetane in an amount of less than about 1 wt. %, alternatively less than about 0.1 wt. %, alternatively less than about 0.01 wt. %, alternatively less than about 0.001 wt. %, alternatively less than about 0.0001 wt. %, alternatively less than about 0.00001 wt. %, or alternatively less than about 0.000001 wt. %, based on the total weight of the reaction mixture. In such embodiment, referring to the CAHM characterized by Structure VII and/or to the CAHM characterized by Structure VIII, z=0. In such embodiment, the CAHM characterized by Structure VII comprises a compound characterized by Structure XXXII, and/or the CAHM characterized by Structure VIII comprises a compound characterized by Structure XXXIII:

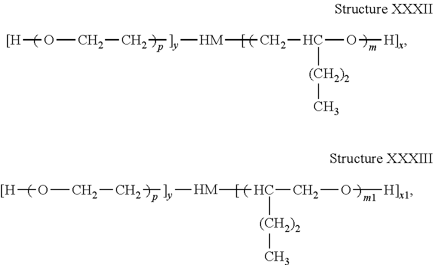

where HM represents the humus material; the repeating methylene (—CH₂—) unit may occur n times with the value of n ranging from about 0 to about 3, alternatively from about 0 to about 2, or alternatively from about 0 to about 1, as previously described for the C3+ cyclic ether compound characterized by Structure III; the repeating C3+ cyclic ether unit that originates from the C3+ cyclic ether in the presence of a strong base catalyst may occur m times with the value of m ranging from about 1 to about 30, alternatively from about 2 to about 20, or alternatively from about 2 to about 10; the repeating C3+ cyclic ether unit that originates from the C3+ cyclic ether (e.g., C3+ epoxide) in the presence of a strong acid catalyst may occur m1 times with the value of m1 ranging from about 1 to about 30, alternatively from about 2 to about 20, or alternatively from about 2 to about 10; the C3+ alkoxylating element may occur x times with the value of x ranging from about 1 to about 300, alternatively from about 2 to about 250, or alternatively from about 10 to about 200, per 100 g of humus material; the C3+ alkoxylating element may occur x1 times with the value of x1 ranging from about 1 to about 300, alternatively from about 2 to about 250, or alternatively from about 10 to about 200, per 100 g of humus material; the repeating ethoxy unit (e.g., when the optional ethylene oxide is used in the alkoxylation along with the C3+ cyclic ether) may occur p times with the value of p ranging from about 1 to about 30, alternatively from about 2 to about 20, or alternatively from about 2 to about 10; and the ethoxylating element may occur y times with the value of y ranging from about 1 to about 200, alternatively from about 1 to about 150, or alternatively from about 2 to about 100, per 100 g of humus material.

In an embodiment, the reaction mixture comprises a strong base catalyst and optionally ethylene oxide along with the C3+ cyclic ether, as previously described herein. In such embodiment, referring to the CAHM characterized by Structure XXXII, y≠0. In such embodiment, the CAHM characterized by Structure XXXII comprises a propoxylated/ethoxylated humus material characterized by Structure XXXIV, a butoxylated/ethoxylated humus material characterized by Structure XXXV, a pentoxylated/ethoxylated humus material characterized by Structure XXXVI, and the like, or combinations thereof.

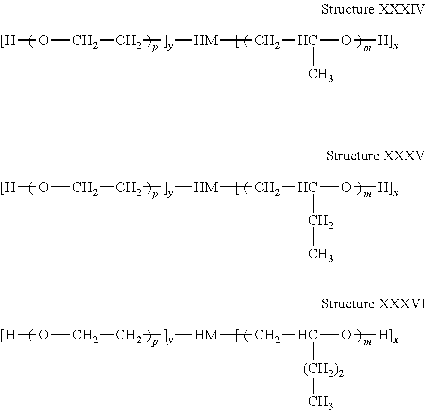

In an embodiment, the reaction mixture comprises a strong acid catalyst and optionally ethylene oxide along with the C3+ cyclic ether, as previously described herein. In such embodiment, referring to the CAHM characterized by Structure XXXIII, y≠0. In such embodiment, the CAHM characterized by Structure XXXIII comprises a propoxylated/ethoxylated humus material characterized by Structure XXXVII, a butoxylated/ethoxylated humus material characterized by Structure XXXVIII, a pentoxylated/ethoxylated humus material characterized by Structure XXXIX, and the like, or combinations thereof.

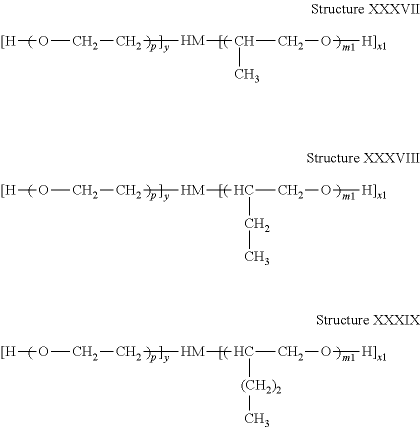

In an embodiment, the reaction mixture comprises a humus material, a C3+ cyclic ether, a strong base catalyst and an inert reaction solvent. For example, the reaction mixture may comprise 4 wt. % leonardite comprising less than about 2 wt. % water based on the weight of the leonardite, propylene oxide as characterized by Structure IV in a weight ratio of propylene oxide to leonardite of 25:1, 50 wt. % sodium methoxide based on the weight of the leonardite, and the balance comprises xylene. The reaction mixture may be heated at a temperature of about 150° C. for about 4 h in a substantially oxygen-free atmosphere (e.g., under a nitrogen atmosphere). In an embodiment, the recovered CAHM comprises a solid propoxylated leonardite (e.g., a compound characterized by Structure XIX), where the value of m is about 25, and the value of x is about 1.

In an embodiment, the reaction mixture comprises a humus material, a C3+ cyclic ether, a strong base catalyst, an inert reaction solvent, and ethylene oxide. For example, the reaction mixture may comprise 4 wt. % CARBONOX filtration control agent comprising less than about 2 wt. % water based on the weight of the CARBONOX filtration control agent, propylene oxide as characterized by Structure IV in a weight ratio of propylene oxide to CARBONOX filtration control agent of 10:1, ethylene oxide in a weight ratio of ethylene oxide to CARBONOX filtration control agent of 15:1, 50 wt. % sodium methoxide based on the weight of the CARBONOX filtration control agent, and the balance comprises xylene. The reaction mixture may be heated at a temperature of about 150° C. for about 8 h in a substantially oxygen-free atmosphere (e.g., under a nitrogen atmosphere). In an embodiment, the recovered CAHM comprises a solid a propoxylated/ethoxylated CARBONOX filtration control agent (e.g., a compound characterized by Structure XXXIV), where the value of m is about 2, the value of x is about 15, the value of p is about 1.2, and the value of y is about 10.

In an embodiment, the reaction mixture comprises a humus material, a C3+ cyclic ether, a strong acid catalyst and an inert reaction solvent. For example, the reaction mixture may comprise 4 wt. % CARBONOX filtration control agent comprising less than about 2 wt. % water based on the weight of the CARBONOX filtration control agent, propylene oxide as characterized by Structure IV in a weight ratio of propylene oxide to CARBONOX filtration control agent of 15:1, oxetane as characterized by Structure II in a weight ratio of oxetane to CARBONOX filtration control agent of 10:1, 2 wt. % HF/(CH₃O)₃Al based on the weight of the CARBONOX filtration control agent, and the balance comprises xylene. The reaction mixture may be heated at a temperature of about 150° C. for about 7 h in a substantially oxygen-free atmosphere (e.g., under a nitrogen atmosphere). In an embodiment, the recovered CAHM comprises a solid propoxylated to CARBONOX filtration control agent (e.g., a compound characterized by Structure XIV), where the value of m1 is about 4, the value of x1 is about 15, the value of q is about 3, and the value of y is about 10.

In an embodiment, the C3+ alkoxylated humus materials (CAHMs) and methods of making same disclosed herein present the advantage of employing naturally-occurring materials (e.g., humus materials) that are widely-available and cost effective, thereby rendering the CAHMs cost effective.

In an embodiment, the CAHMs disclosed herein may be produced with a wide range of properties, such as for example variable solubility in different types of solvents (e.g., polar solvents, water, polar organic solvents, methanol, aromatic hydrocarbon solvents, xylene, petroleum oil, alkane hydrocarbons, pentane, etc.), based on the ratio between the C3+ cyclic ether and humus material used in the reaction mixture, and also based on the reaction conditions. The variable solubility of different CAHMs in different types of solvents may advantageously allow the CAHMs to exhibit different surface active behavior based on the particular composition of the CAHM.

In an embodiment, the CAHMs disclosed herein may advantageously exhibit an elevated tolerance to salinity and pH. For example, the CAHMs may be used in fluids comprising salts in an amount of from about 0.1 wt. % to about 20 wt. %, alternatively about 0.1 wt. % to about 5 wt. %, alternatively from about 5 wt. % to about 10 wt. %, or alternatively from about 10 wt. % to about 20 wt. %, based on the weight of the fluid. For example, the CAHMs may be used in fluids comprising a pH in the range of from about 2 to about 12, alternatively from about 7 to about 11, or alternatively from about 8 to about 10.

In an embodiment, the CAHMs disclosed herein may advantageously exhibit a high temperature stability, owing to the inherent high temperature stability of the humus materials. For example, the CAHMs may be used in environments comprising a temperature in the range of from about 20° C. to about 260° C., alternatively from about 20° C. to about 177° C., or alternatively from about 20° C. to about 121° C.

In an embodiment, the CAHMs disclosed herein may be advantageously employed in a variety of applications, such as for example in a wellbore servicing operation. In an embodiment, the CAHMs may be advantageously used as additives, such as for example surfactants, viscosifiers, suspension agents, rheology control agents, deflocculants, lubricants, mud lubricants, torque and drag reduction agents, fluid loss control agents, mud dispersants, and the like, in fluids and compositions suitable for wellbore servicing operations.

### ADDITIONAL DISCLOSURE

A first embodiment, which is a method of alkoxylating a humus material comprising:

heating a reaction mixture comprising a humus material, a C3+ cyclic ether, a catalyst and an inert reaction solvent; and

recovering a C3+ alkoxylated humus material from the reaction mixture.

A second embodiment, which is the method of the first embodiment wherein the reaction mixture is heated to a temperature of from about 130° C. to about 170° C.

A third embodiment, which is the method of any of the first through the second embodiments wherein the humus material, the catalyst and the inert reaction solvent are pre-mixed prior to the addition of the C3+ cyclic ether.

A fourth embodiment, which is the method of any of the first through the third embodiments wherein the reaction mixture is heated in a substantially oxygen-free atmosphere.

A fifth embodiment, which is the method of any of the first through the fourth embodiments wherein the humus material comprises brown coal, lignite, subbituminous coal, leonardite, humic acid, a compound characterized by Structure I, fulvic acid, humin, peat, lignin, or combinations thereof.

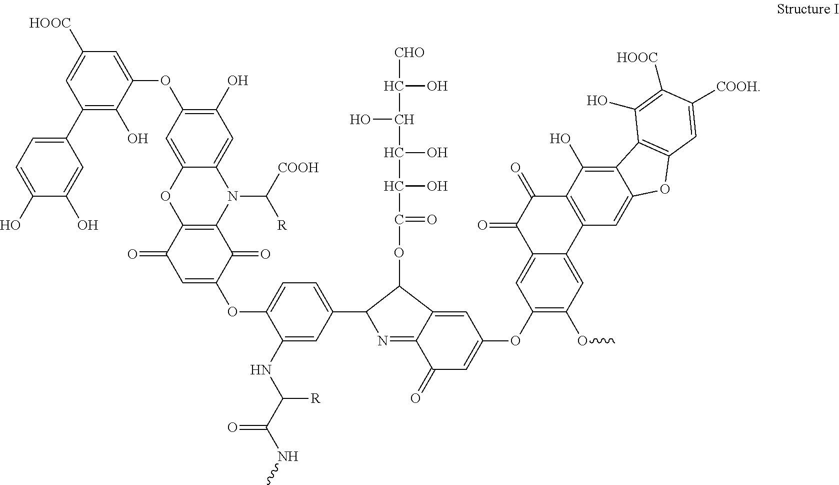

A sixth embodiment, which is the method of any of the first through the fifth embodiments wherein the humus material comprises less than about 3.5 wt. % water based on the total weight of the humus material.

A seventh embodiment, which is the method of any of the first through the sixth embodiments wherein the humus material comprises a particle size such that equal to or greater than about 97 wt. % passes through an about 80 mesh screen (U.S. Sieve Series) and equal to or greater than about 55 wt. % passes through an about 200 mesh screen (U.S. Sieve Series).

An eighth embodiment, which is the method of any of the first through the seventh embodiments wherein the humus material is present in the reaction mixture in an amount of from about 1 wt. % to about 50 wt. % based on the total weight of the reaction mixture.

A ninth embodiment, which is the method of any of the first through the eighth embodiments wherein the C3+ cyclic ether comprises oxetane as characterized by Structure II, a C3+ epoxide compound characterized by Structure III, or combinations thereof,

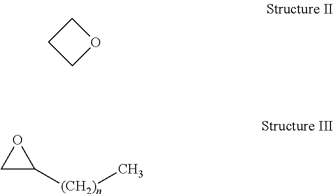

wherein the repeating methylene (—CH₂—) unit may occur n times with the value of n ranging from about 0 to about 3.

A tenth embodiment, which is the method of the ninth embodiment wherein the C3+ epoxide compound characterized by Structure III comprises propylene oxide as characterized by Structure IV, butylene oxide as characterized by Structure V, pentylene oxide as characterized by Structure VI, or combinations thereof.

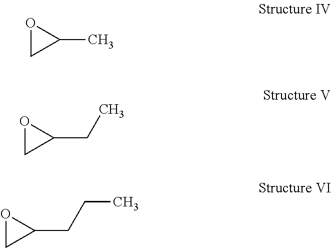

An eleventh embodiment, which is the method of any of the first through the tenth embodiments wherein the C3+ cyclic ether is present in the reaction mixture in a weight ratio of C3+ cyclic ether to humus material of from about 0.5:1 to about 50:1.

A twelfth embodiment, which is the method of any of the first through the eleventh embodiments wherein the catalyst comprises a strong base catalyst.

A thirteenth embodiment, which is the method of the twelfth embodiment wherein the strong base catalyst comprises sodium methoxide, potassium methoxide, sodium ethoxide, potassium ethoxide, or combinations thereof.

A fourteenth embodiment, which is the method of any of the twelfth through the thirteenth embodiments wherein the strong base catalyst is present in the reaction mixture in an amount of from about 0.1 wt. % to about 75 wt. % based on the total weight of the humus material.

A fifteenth embodiment, which is the method of any of the first through the eleventh embodiments wherein the catalyst comprises a strong acid catalyst.

A sixteenth embodiment, which is the method of the fifteenth embodiment wherein the strong acid catalyst comprises a mixture of HF and a metal alkoxide and/or a mixed metal alkoxide; or a mixture of esters of titanic and/or zirconic acid with monoalkanols and sulfuric acid and/or alkanesulfonic acids and/or aryloxysulfonic acids.

A seventeenth embodiment, which is the method of any of the fifteenth through the sixteenth embodiments wherein the strong acid catalyst is present in the reaction mixture in an amount of from about 0.01 wt. % to about 10 wt. % based on the total weight of the humus material.

An eighteenth embodiment, which is the method of any of the first through the seventeenth embodiments wherein the inert reaction solvent comprises C₆-C₁₂ liquid aromatic hydrocarbons.

A nineteenth embodiment, which is the method of the eighteenth embodiment wherein the C₆-C₁₂ liquid aromatic hydrocarbons comprise toluene, ethylbenzene, xylenes, o-xylene, m-xylene, p-xylene, trimethylbenzenes, cumene, mesitylene, 1,2,4-trimethylbenzene, 1,2,3-trimethylbenzene, or combinations thereof.

A twentieth embodiment, which is the method of any of the first through the nineteenth embodiments wherein the inert reaction solvent is present in the reaction mixture in an amount of from about 30 wt. % to about 90 wt. % based on the total weight of the reaction mixture.

A twenty-first embodiment, which is the method of any of the first through the twentieth embodiments wherein the reaction mixture further comprises ethylene oxide.

A twenty-second embodiment, which is the method of the twenty-first embodiment wherein the weight ratio of ethylene oxide to C3+ cyclic ether is in the range of from about 10:1 to about 1:10.

A twenty-third embodiment, which is the method of any of the first through the fourteenth and the eighteenth through the twenty-second embodiments wherein the catalyst comprises a strong base catalyst and the C3+ alkoxylated humus material comprises a compound characterized by Structure VII:

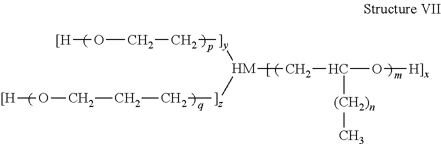

wherein HM represents the humus material; n is in the range of from about 0 to about 3; m is in the range of from about 1 to about 30; x is in the range of from about 0 to about 300, per 100 g of humus material; p is in the range of from about 1 to about 30; y is in the range of from about 0 to about 200, per 100 g of humus material; q is in the range of from about 1 to about 30; z is in the range of from about 0 to about 300, per 100 g of humus material; and x and z cannot both be 0 at the same time.

A twenty-fourth embodiment, which is the method of any of the first through the eleventh and the fifteen through the twenty-second embodiments wherein the catalyst comprises a strong acid catalyst and the C3+ alkoxylated humus material comprises a compound characterized by Structure VIII:

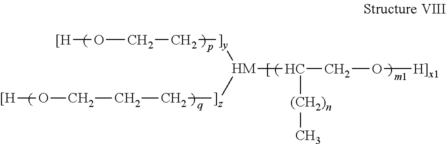

wherein HM represents the humus material; n is in the range of from about 0 to about 3; m1 is in the range of from about 1 to about 30; x1 is in the range of from about 0 to about 300, per 100 g of humus material; p is in the range of from about 1 to about 30; y is in the range of from about 0 to about 200, per 100 g of humus material; q is in the range of from about 1 to about 30; z is in the range of from about 0 to about 300, per 100 g of humus material; and x1 and z cannot both be 0 at the same time.

A twenty-fifth embodiment, which is a C3+ alkoxylated humus material produced by the method of any of the first through the twenty-fourth embodiments.

A twenty-sixth embodiment, which is the C3+ alkoxylated humus material of the twenty-fifth embodiment wherein the reaction mixture comprises ethylene oxide.

A twenty-seventh embodiment, which is a method of alkoxylating a humus material comprising:

heating a reaction mixture comprising a humus material, a C3+ cyclic ether, a catalyst and an inert reaction solvent to a temperature of from about 130° C. to about 170° C., wherein the humus material comprises leonardite, the C3+ cyclic ether comprises propylene oxide, and the inert reaction solvent comprises xylene; and

recovering a C3+ alkoxylated humus material from the reaction mixture.

A twenty-eighth embodiment, which is the method of the twenty-seventh embodiment wherein the reaction mixture is heated in a substantially oxygen-free atmosphere.

A twenty-ninth embodiment, which is the method of any of the twenty-seventh through the twenty-eighth embodiments wherein the reaction mixture comprises ethylene oxide, the catalyst comprises a strong base catalyst, and the C3+ alkoxylated humus material comprises a propoxylated/ethoxylated humus material characterized by Structure XXXIV:

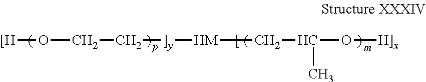

wherein HM represents the humus material; m is in the range of from about 1 to about 30; x is in the range of from about 1 to about 300, per 100 g of humus material; p is in the range of from about 1 to about 20; and y is in the range of from about 1 to about 200, per 100 g of humus material.

A thirtieth embodiment, which is the method of any of the twenty-seventh through the twenty-eighth embodiments wherein the reaction mixture comprises ethylene oxide, the catalyst comprises a strong acid catalyst, and the C3+ alkoxylated humus material comprises a propoxylated/ethoxylated humus material characterized by Structure XXXVII:

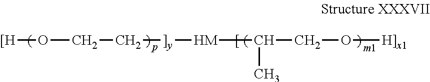

wherein HM represents the humus material; m1 is in the range of from about 1 to about 30; x1 is in the range of from about 1 to about 300, per 100 g of humus material; p is in the range of from about 1 to about 30; and y is in the range of from about 1 to about 200, per 100 g of humus material.

A thirty-first embodiment, which is a C3+ alkoxylated humus material.

A thirty-second embodiment, which is the C3+ alkoxylated humus material of the thirty-first embodiment, characterized by Structure VII:

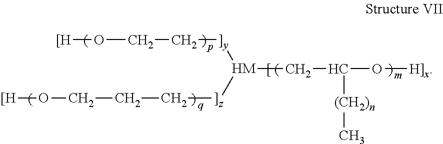

wherein HM represents the humus material; n is in the range of from about 0 to about 3; m is in the range of from about 1 to about 30; x is in the range of from about 0 to about 300, per 100 g of humus material; p is in the range of from about 1 to about 30; y is in the range of from about 0 to about 200, per 100 g of humus material; q is in the range of from about 1 to about 30; z is in the range of from about 0 to about 300, per 100 g of humus material; and x and z cannot both be 0 at the same time.

A thirty-third embodiment, which is the C3+ alkoxylated humus material of the thirty-first embodiment, characterized by Structure VIII:

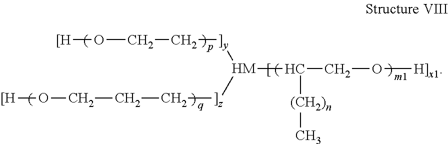

wherein HM represents the humus material; n is in the range of from about 0 to about 3; m1 is in the range of from about 1 to about 30; x1 is in the range of from about 0 to about 300, per 100 g of humus material; p is in the range of from about 1 to about 30; y is in the range of from about 0 to about 200, per 100 g of humus material; q is in the range of from about 1 to about 30; z is in the range of from about 0 to about 300, per 100 g of humus material; and x1 and z cannot both be 0 at the same time.

A thirty-fourth embodiment, which is the C3+ alkoxylated humus material of the thirty-second embodiment wherein y=0.

A thirty-fifth embodiment, which is the C3+ alkoxylated humus material of the thirty-fourth embodiment comprising a compound characterized by Structure IX:

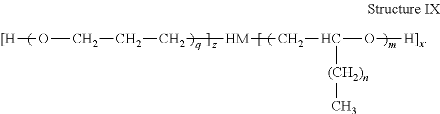

A thirty-sixth embodiment, which is the C3+ alkoxylated humus material of the thirty-fifth embodiment wherein the compound characterized by Structure IX comprises a propoxylated humus material characterized by Structure XI, a propoxylated/butoxylated humus material characterized by Structure XII, a propoxylated/pentoxylated humus material characterized by Structure XIII, or combinations thereof.

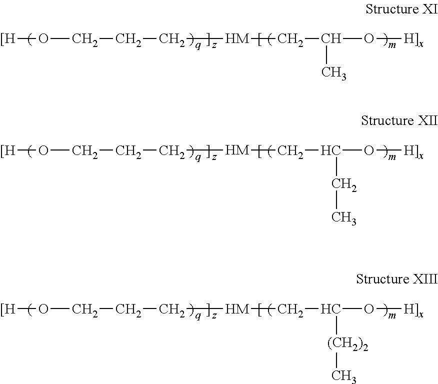

A thirty-seventh embodiment, which is the C3+ alkoxylated humus material of any of the thirty-fifth through the thirty-sixth embodiments wherein z=0.

A thirty-eighth embodiment, which is the C3+ alkoxylated humus material of the thirty-seventh embodiment comprising a compound characterized by Structure XVII:

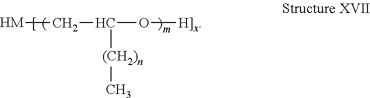

A thirty-ninth embodiment, which is the C3+ alkoxylated humus material of the thirty-eighth embodiment wherein the compound characterized by Structure XVII comprises a propoxylated humus material characterized by Structure XIX, a butoxylated humus material characterized by Structure XX, a pentoxylated humus material characterized by Structure XXI, or combinations thereof.

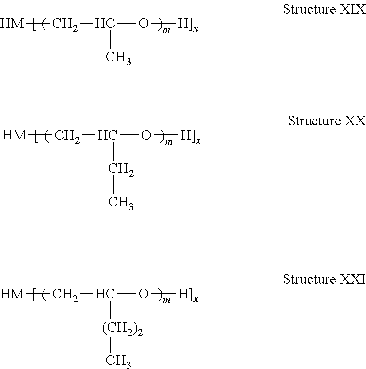

A fortieth embodiment, which is the C3+ alkoxylated humus material of the thirty-third embodiment wherein y=0.

A forty-first embodiment, which is the C3+ alkoxylated humus material of the fortieth embodiment comprising a compound characterized by Structure X:

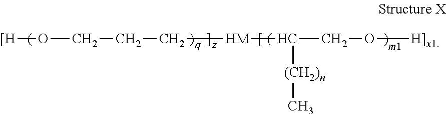

A forty-second embodiment, which is the C3+ alkoxylated humus material of the forty-first embodiment wherein the compound characterized by Structure X comprises a propoxylated humus material characterized by Structure XIV, a propoxylated/butoxylated humus material characterized by Structure XV, a propoxylated/pentoxylated humus material characterized by Structure XVI, or combinations thereof.

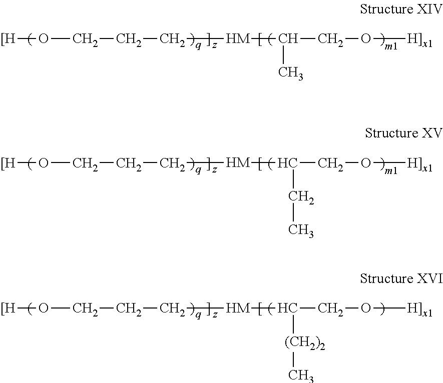

A forty-third embodiment, which is the C3+ alkoxylated humus material of the forty-first or the forty-second embodiment wherein z=0.

A forty-fourth embodiment, which is the C3+ alkoxylated humus material of the forty-third embodiment comprising a compound characterized by Structure XVIII:

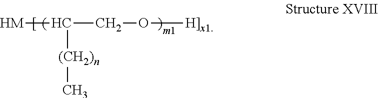

A forty-fifth embodiment, which is the C3+ alkoxylated humus material of the forty-fourth embodiment wherein the compound characterized by Structure XVIII comprises a propoxylated humus material characterized by Structure XXII, a butoxylated humus material characterized by Structure XXIII, a pentoxylated humus material characterized by Structure XXIV, or combinations thereof.

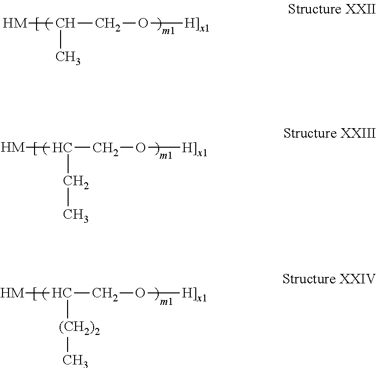

A forty-sixth embodiment, which is the C3+ alkoxylated humus material of the thirty-first embodiment comprising a propoxylated humus material characterized by Structure XXV:

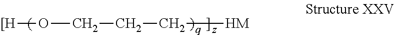

wherein q is in the range of from about 1 to about 30; and z is in the range of from about 1 to about 300, per 100 g of humus material.

A forty-seventh embodiment, which is the C3+ alkoxylated humus material of the thirty-second embodiment wherein the compound characterized by Structure VII comprises a propoxylated/ethoxylated humus material characterized by Structure XXVI, a butoxylated/propoxylated/ethoxylated humus material characterized by Structure XXVII, a pentoxylated/propoxylated/ethoxylated humus material characterized by Structure XXVIII, or combinations thereof.

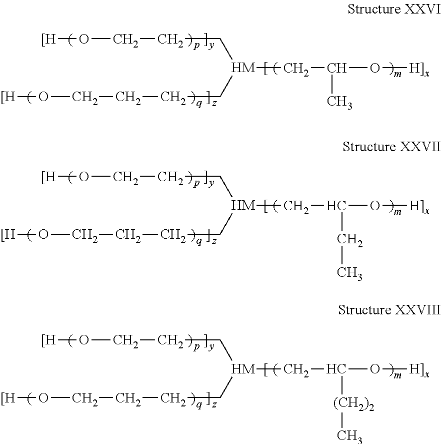

A forty-eighth embodiment, which is the C3+ alkoxylated humus material of the thirty-second embodiment wherein z=0.

A forty-ninth embodiment, which is the C3+ alkoxylated humus material of the forty-eighth embodiment comprising a compound characterized by Structure XXXII:

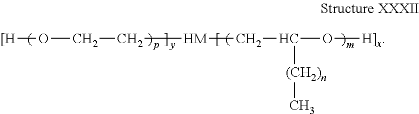

A fiftieth embodiment, which is the C3+ alkoxylated humus material of the forty-ninth embodiment wherein the compound characterized by Structure XXXII comprises a propoxylated/ethoxylated humus material characterized by Structure XXXIV, a butoxylated/ethoxylated humus material characterized by Structure XXXV, a pentoxylated/ethoxylated humus material characterized by Structure XXXVI, or combinations thereof.

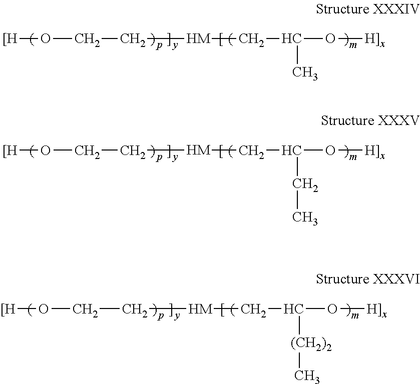

A fifty-first embodiment, which is the C3+ alkoxylated humus material of the thirty-third embodiment wherein the compound characterized by Structure VIII comprises a propoxylated/ethoxylated humus material characterized by Structure XXIX, a butoxylated/propoxylated/ethoxylated humus material characterized by Structure XXX, a pentoxylated/propoxylated/ethoxylated humus material characterized by Structure XXXI, or combinations thereof.

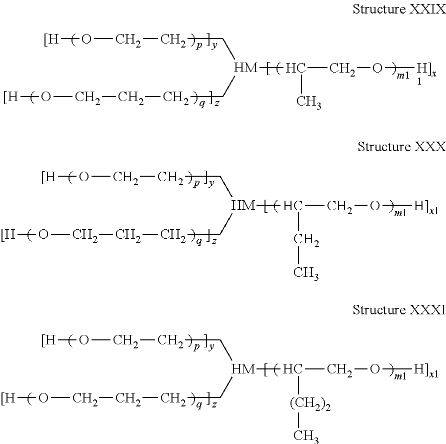

A fifty-second embodiment, which is the C3+ alkoxylated humus material of the thirty-third embodiment wherein z=0.

A fifty-third embodiment, which is the C3+ alkoxylated humus material of the fifty-second embodiment comprising a compound characterized by Structure XXXIII:

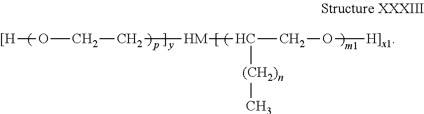

A fifty-fourth embodiment, which is the C3+ alkoxylated humus material of the fifty-third embodiment wherein the compound characterized by Structure XXXIII comprises a propoxylated/ethoxylated humus material characterized by Structure XXXVII, a butoxylated/ethoxylated humus material characterized by Structure XXXVIII, a pentoxylated/ethoxylated humus material characterized by Structure XXXIX, or combinations thereof.

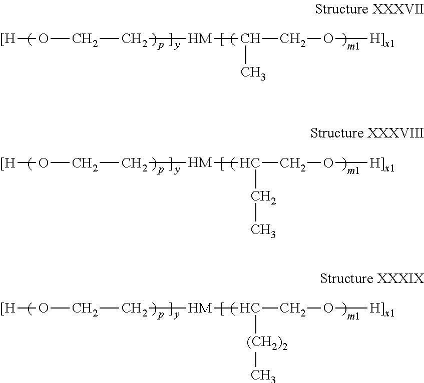

While embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R_{L}, and an upper limit, R_{U}, is disclosed, any number falling within the range is specifically disclosed. In particular, the following numbers within the range are specifically disclosed: R=R_{L}+k*(R_{U}−R_{L}), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A method of alkoxylating a humus material comprising:
heating a reaction mixture comprising a humus material, a C3+ cyclic ether, a catalyst and an inert reaction solvent; and
recovering a C3+ alkoxylated humus material from the reaction mixture.

2-4. (canceled)

5. The method of claim 1 wherein the humus material comprises brown coal, lignite, subbituminous coal, leonardite, humic acid, a compound characterized by Structure I, fulvic acid, humin, peat, lignin, or combinations thereof.

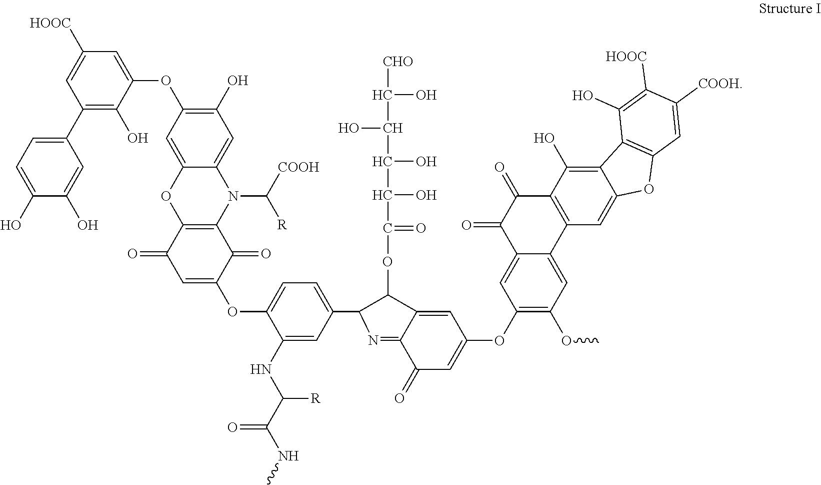

6-8. (canceled)

9. The method of claim 1 wherein the C3+ cyclic ether comprises oxetane as characterized by Structure II, a C3+ epoxide compound characterized by Structure III, or combinations thereof, 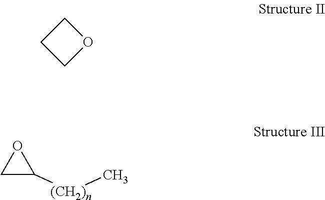
wherein the repeating methylene (—CH₂—) unit may occur n times with the value of n ranging from about 0 to about 3.

10. The method of claim 9 wherein the C3+ epoxide compound characterized by Structure III comprises propylene oxide as characterized by Structure IV, butylene oxide as characterized by Structure V, pentylene oxide as characterized by Structure VI, or combinations thereof.

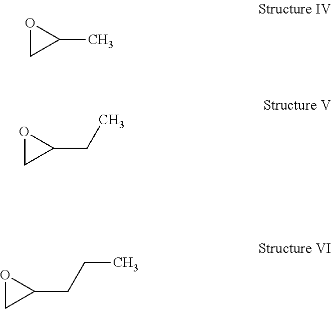

11-12. (canceled)

13. The method of claim 23 wherein the strong base catalyst comprises sodium methoxide, potassium methoxide, sodium ethoxide, potassium ethoxide, or combinations thereof.

14-15. (canceled)

16. The method of claim 23 wherein the strong acid catalyst comprises (a) a mixture of HF and at least one of a metal alkoxide and a mixed metal alkoxide; or (b) a mixture of esters of at least one of titanic and zirconic acid with monoalkanols and at least one of sulfuric acid, alkanesulfonic acids and aryloxysulfonic acids.

18. The method of claim 1 wherein the inert reaction solvent comprises C₆-C₁₂ liquid aromatic hydrocarbons.

19. The method of claim 18 wherein the C₆-C₁₂ liquid aromatic hydrocarbon is selected from the group consisting of toluene, ethylbenzene, xylenes, o-xylene, m-xylene, p-xylene, trimethylbenzenes, cumene, mesitylene, 1,2,4-trimethylbenzene, and 1,2,3-trimethylbenzene.

20. (canceled)

21. The method of claim 1 wherein the reaction mixture further comprises ethylene oxide.

22. (canceled)

23. The method of claim 1 wherein the catalyst is selected from the group consisting of a strong base catalyst and a strong acid catalyst, wherein:
(a) when the catalyst is a strong base catalyst, the C3+ alkoxylated humus material comprises a compound characterized by Structure VII: 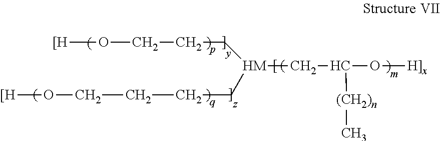
wherein HM represents the humus material; n is in the range of from about 0 to about 3; m is in the range of from about 1 to about 30; x is in the range of from about 0 to about 300, per 100 g of humus material; p is in the range of from about 1 to about 30; y is in the range of from about 0 to about 200, per 100 g of humus material; q is in the range of from about 1 to about 30; z is in the range of from about 0 to about 300, per 100 g of humus material; and x and z cannot both be 0 at the same time; or
(b) when the catalyst is a strong acid catalyst, the C3+ alkoxylated humus material comprises a compound characterized by Structure VIII: 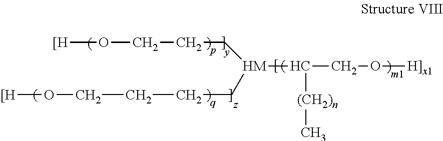
wherein HM represents the humus material; n is in the range of from about 0 to about 3; m1 is in the range of from about 1 to about 30; x1 is in the range of from about 0 to about 300, per 100 g of humus material; p is in the range of from about 1 to about 30; y is in the range of from about 0 to about 200, per 100 g of humus material; q is in the range of from about 1 to about 30; z is in the range of from about 0 to about 300, per 100 g of humus material; and x1 and z cannot both be 0 at the same time.

24-26. (canceled)

27. A method of alkoxylating a humus material comprising:
heating a reaction mixture comprising a humus material, a C3+ cyclic ether, a catalyst and an inert reaction solvent to a temperature of from about 130° C. to about 170° C., wherein the humus material comprises leonardite, the C3+ cyclic ether comprises propylene oxide, and the inert reaction solvent comprises xylene; and
recovering a C3+ alkoxylated humus material from the reaction mixture.

28. (canceled)

29. The method of claim 27 wherein:
the reaction mixture comprises ethylene oxide and the catalyst is selected from the group consisting of a strong base catalyst and a strong acid catalyst, wherein,
(a) when the catalyst comprises a strong base catalyst, the C3+ alkoxylated humus material comprises a propoxylated/ethoxylated humus material characterized by Structure XXXIV: 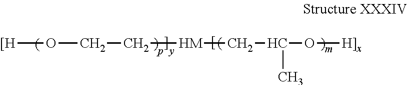
wherein HM represents the humus material; m is in the range of from about 1 to about 30; x is in the range of from about 1 to about 300, per 100 g of humus material; p is in the range of from about 1 to about 20; and y is in the range of from about 1 to about 200, per 100 g of humus material; or
(b) when the catalyst comprises a strong acid catalyst, the C3+ alkoxylated humus material comprises a propoxylated/ethoxylated humus material characterized by Structure XXXVII: 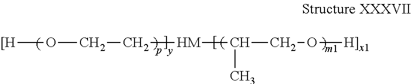
wherein HM represents the humus material; m1 is in the range of from about 1 to about 30; x1 is in the range of from about 1 to about 300, per 100 g of humus material; p is in the range of from about 1 to about 30; and y is in the range of from about 1 to about 200, per 100 g of humus material.

30. (canceled)

31. A C3+ alkoxylated humus material.

32. The C3+ alkoxylated humus material of claim 31 characterized by:
(a) Structure VII: 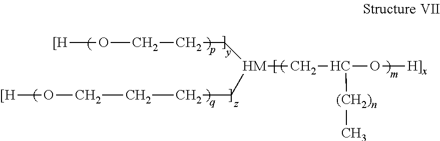
wherein HM represents the humus material; n is in the range of from about 0 to about 3; m is in the range of from about 1 to about 30; x is in the range of from about 0 to about 300, per 100 g of humus material; p is in the range of from about 1 to about 30; y is in the range of from about 0 to about 200, per 100 g of humus material; q is in the range of from about 1 to about 30; z is in the range of from about 0 to about 300, per 100 g of humus material; and x and z cannot both be 0 at the same time; or
(b) Structure VIII: 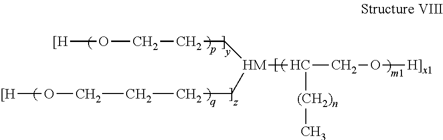
wherein HM represents the humus material; n is in the range of from about 0 to about 3; m1 is in the range of from about 1 to about 30; x1 is in the range of from about 0 to about 300, per 100 g of humus material; p is in the range of from about 1 to about 30; y is in the range of from about 0 to about 200, per 100 g of humus material; q is in the range of from about 1 to about 30; z is in the range of from about 0 to about 300, per 100 g of humus material; and x1 and z cannot both be 0 at the same time.

33-35. (canceled)

36. The C3+ alkoxylated humus material of claim 32 wherein y=0 and wherein:
(a) the compound characterized by Structure VII comprises a propoxylated humus material characterized by Structure XI, a propoxylated/butoxylated humus material characterized by Structure XII, a propoxylated/pentoxylated humus material characterized by Structure XIII, or combinations thereof 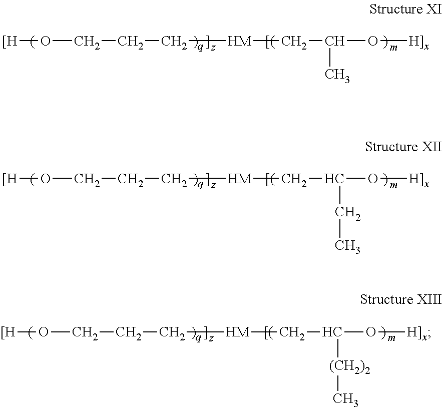
and
(b) wherein the compound characterized by Structure VIII comprises a propoxylated humus material characterized by Structure XIV, a propoxylated/butoxylated humus material characterized by Structure XV, a propoxylated/pentoxylated humus material characterized by Structure XVI, or combinations thereof

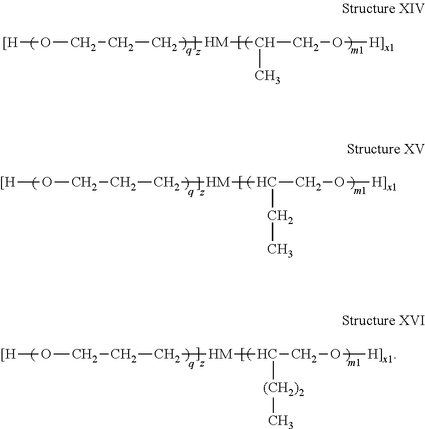

37-38. (canceled)

39. The C3+ alkoxylated humus material of claim 36 wherein z=0 and wherein:
(a) the compound characterized by Structure VII comprises a propoxylated humus material characterized by Structure XIX, a butoxylated humus material characterized by Structure XX, a pentoxylated humus material characterized by Structure XXI, or combinations thereof 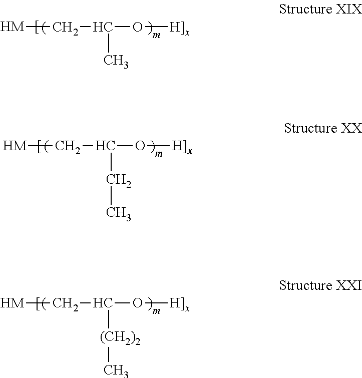
and
(b) wherein the compound characterized by Structure VIII comprises a propoxylated humus material characterized by Structure XXII, a butoxylated humus material characterized by Structure XXIII, a pentoxylated humus material characterized by Structure XXIV, or combinations thereof

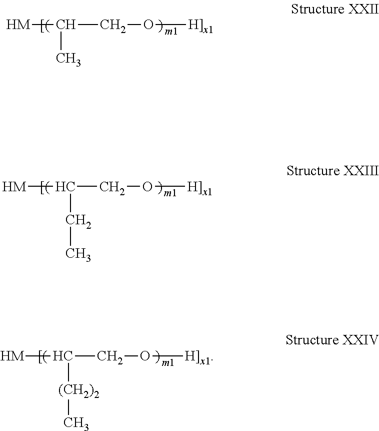

40-45. (canceled)

46. The C3+ alkoxylated humus material of claim 31 comprising a propoxylated humus material characterized by Structure XXV: 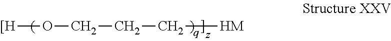
wherein q is in the range of from about 1 to about 30; and z is in the range of from about 1 to about 300, per 100 g of humus material.

47. The C3+ alkoxylated humus material of claim 32 wherein the compound characterized by Structure VII comprises a propoxylated/ethoxylated humus material characterized by Structure XXVI, a butoxylated/propoxylated/ethoxylated humus material characterized by Structure XXVII, a pentoxylated/propoxylated/ethoxylated humus material characterized by Structure XXVIII, or combinations thereof

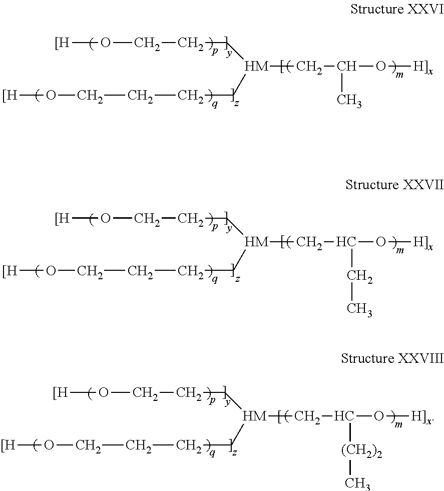

48-49. (canceled)

50. The C3+ alkoxylated humus material of claim 32 wherein z=0 and wherein:
(a) the compound characterized by Structure VII comprises a propoxylated/ethoxylated humus material characterized by Structure XXXIV, a butoxylated/ethoxylated humus material characterized by Structure XXXV, a pentoxylated/ethoxylated humus material characterized by Structure XXXVI, or combinations thereof 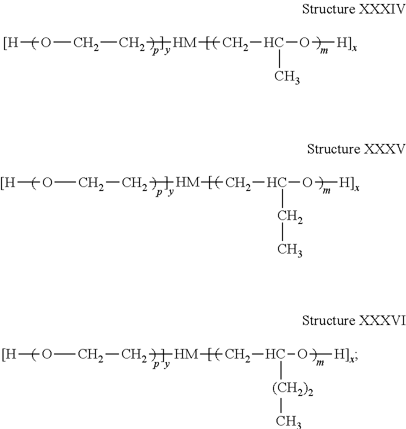
and
(b) wherein the compound characterized by Structure VIII comprises a propoxylated/ethoxylated humus material characterized by Structure XXXVII, a butoxylated/ethoxylated humus material characterized by Structure XXXVIII, a pentoxylated/ethoxylated humus material characterized by Structure XXXIX, or combinations thereof

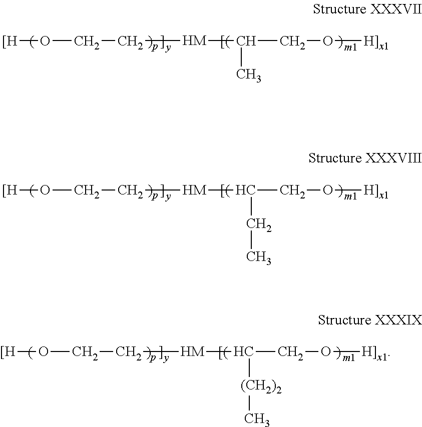

51. The C3+ alkoxylated humus material of claim 32 wherein the compound characterized by Structure VIII comprises a propoxylated/ethoxylated humus material characterized by Structure XXIX, a butoxylated/propoxylated/ethoxylated humus material characterized by Structure XXX, a pentoxylated/propoxylated/ethoxylated humus material characterized by Structure XXXI, or combinations thereof

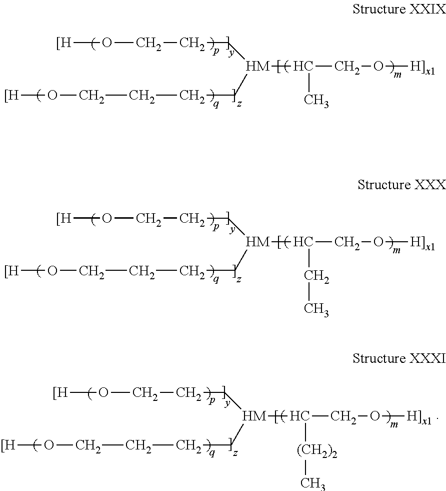

52-54. (canceled)
